(19) United States
(12) Patent Application Publication
(10) Pub. No.: US 2020/0002665 A1
(43) Pub. Date: Jan. 2, 2020

(54) Methods of Applying Ammonia Toxicity and Inducing Nitrogen Uptake in Microalgae Cultures

(71) Applicant: Heliae Development, LLC, Gilbert, AZ (US)

(72) Inventors: Eneko Ganuza Taberna, Tempe, AZ (US); Charles Sellers, Mesa, AZ (US); Kara Bautista, Menlo Park, CA (US)

(21) Appl. No.: 16/467,703

(22) PCT Filed: Dec. 14, 2017
(86) PCT No.: PCT/US17/66314

§ 371 Date: Jun. 7, 2019

Related U.S. Application Data
(60) Provisional application No. 62/434,030, filed on Dec. 14, 2016.

(51) Int. Cl. C12N 1/12; A01G 33/00; C12M 1/00; C12M 1/34

(52) U.S. Cl. CPC C12N 1/12; C12M 41/32; C12M 21/02; A01G 33/00

(57) ABSTRACT

Methods for culturing microalgae in ammonia or ammonium toxicity conditions to induce the uptake of nitrogen, increase the metabolic rate, and increase the accumulation of protein, are disclosed. Embodiments include methods of controlling the internal microalgae cell ammonium concentration by manipulating the culture pH and residual ammonia or ammonium concentration.

### CROSS-REFERENCE TO RELATED APPLICATIONS

This application is a continuation of and claims the benefit of U.S. Provisional Application Ser. No. 62/434,030 filed on Dec. 14, 2016. The entirety of such application is incorporated herein by reference.

### BACKGROUND

The use of ammonium and ammonia as a nitrogen source for microalgae enables the culture to experience the high productivities associated with mixotrophic and heterotrophic cultures. However, over time the residual ammonia or ammonium concentration of the microalgae can rise to levels that are toxic to the microalgae without careful control. Industrial cultivation of microalgae also requires optimization of the conditions for growth and accumulation of target metabolites for efficient commercial production. For example, microalgae enriched with protein are desirable for the nutrition, food, and feed markets. A thorough understanding of the microalgae cells metabolism and the interaction between nitrogen uptake, toxicity, cell growth, and metabolite accumulation, may dictate which methods, conditions, and inputs to use for commercial production.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

As disclosed herein, methods and techniques of culturing microalgae in ammonia or ammonium toxicity conditions in order to produce a benefit for the microalgae culture. Embodiments can comprise inducing the uptake of ammonium; increasing the metabolic rate; and increasing the accumulation of protein. Embodiments include methods of controlling the internal microalgae cell ammonium concentration by manipulating the culture pH and residual ammonium or ammonia concentration.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

The inventive concepts described herein may take physical form in certain parts and arrangements of parts, a preferred embodiment of which will be described in detail in the specification and illustrated in the accompanying drawings which form a part hereof, and wherein:

FIG. 1 illustrates an exemplary block diagram of a system, according to an embodiment.

FIG. 2 illustrates a schematic side view of a system, according to an embodiment.

FIG. 3 illustrates an exemplary block diagram of a system, according to an embodiment.

FIG. 4 illustrates a system, according to an embodiment.

FIG. 5 illustrates a perspective view of an exemplary modular bioreactor system embodiment with modules that can be coupled and decoupled.

FIG. 6 illustrates a perspective view of an exemplary cascading transfer bioreactor system embodiment.

FIG. 7 illustrates a perspective view of an open raceway pond bioreactor embodiment with turning vanes and thrusters.

FIG. 8 shows a diagram of ammonia and ammonium uptake in a cell.

FIG. 9 shows a representation of the results of uptake and assimilation of different nitrogen sources.

FIG. 10 shows a graph of the change in culture pH for cultures comprising different nitrogen sources.

FIG. 11 shows a graph of the growth and productivity of microalgae with different concentrations of different nitrogen sources.

FIG. 12 shows a representation of the titrant pulses in an auxostat system utilizing acetic acid or ammonia hydroxide.

FIG. 13 shows a graph comparing the cell dry weight over time of microalgae cultures grown at different culture pH values.

FIG. 14 shows a graph of the residual ammonia concentration for microalgae cultures grown at different pH values.

FIG. 15 shows a graph of total protein content in microalgae cultures grown at different culture pH values.

FIG. 16 shows the final cell dry weight of microalgae cultures grown at different culture pH values.

FIG. 17 shows the final total protein for microalgae cultures grown at different culture pH values.

FIG. 18 shows a graph comparing the cell dry weight over time for microalgae cultures receiving different sources of nitrogen.

FIG. 19 shows a graph of the residual nitrate level over time in a microalgae culture.

FIG. 20 shows a graph of the residual ammonia level over time in a microalgae culture.

FIG. 21 shows a graph comparing the total protein content for microalgae cultures with receiving different sources of nitrogen.

FIG. 22 shows a graph comparing the cell dry weight over time for microalgae cultures receiving different sources of nitrogen and cultured at different pH values.

FIG. 23 shows a graph comparing the residual nitrate level in microalgae cultures with different culture pH values.

FIG. 24 shows a graph comparing the residual ammonia level in microalgae cultures with different culture pH values.

FIG. 25 shows a graph comparing the total protein content of microalgae cultures receiving different sources of nitrogen and cultured at different pH values.

FIG. 26 shows a graph comparing cell dry weight over time for microalgae cultures receiving different sources of nitrogen and cultured at different pH values.

FIG. 27 shows a graph comparing the residual nitrate level in microalgae cultures with different culture pH values.

FIG. 28 shows a graph comparing the residual ammonia level in microalgae cultures with different culture pH values.

FIG. 29 shows a graph comparing the total protein content in microalgae cultures receiving different nitrogen sources and with different culture pH values.

FIG. 30 shows a graph comparing the cell dry weight over time for microalgae cultures receiving different nitrogen sources.

FIG. 31 shows a graph of the residual ammonia level in a microalgae culture.

FIG. 32 shows a graph of the residual glutamate level in a microalgae culture.

FIG. 33 shows a graph comparing the total protein level for microalgae cultures receiving different nitrogen sources.

FIG. 34 is a flow diagram illustrating an exemplary method for culturing microalgae in ammonia or ammonium toxicity conditions in order to produce a benefit for the microalgae culture.

FIG. 35 is a schematic diagram illustrating an exemplary system for culturing microalgae in ammonia or ammonium toxicity conditions in order to produce a benefit for the microalgae culture.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

With reference to the drawings, like reference numerals designate identical or corresponding parts throughout the several views. However, the inclusion of like elements in different views does not mean a given embodiment necessarily includes such elements or that all embodiments of the inventive concepts include such elements. The examples and figures are illustrative only and not meant to limit the inventive concepts, which is measured by the scope and spirit of the claims.

The term “microalgae” refers to microscopic single cell organisms such as microalgae, cyanobacteria, algae, diatoms, dinoflagellates, freshwater organisms, marine organisms, or other similar single cell organisms capable of growth in phototrophic, mixotrophic, or heterotrophic culture conditions.

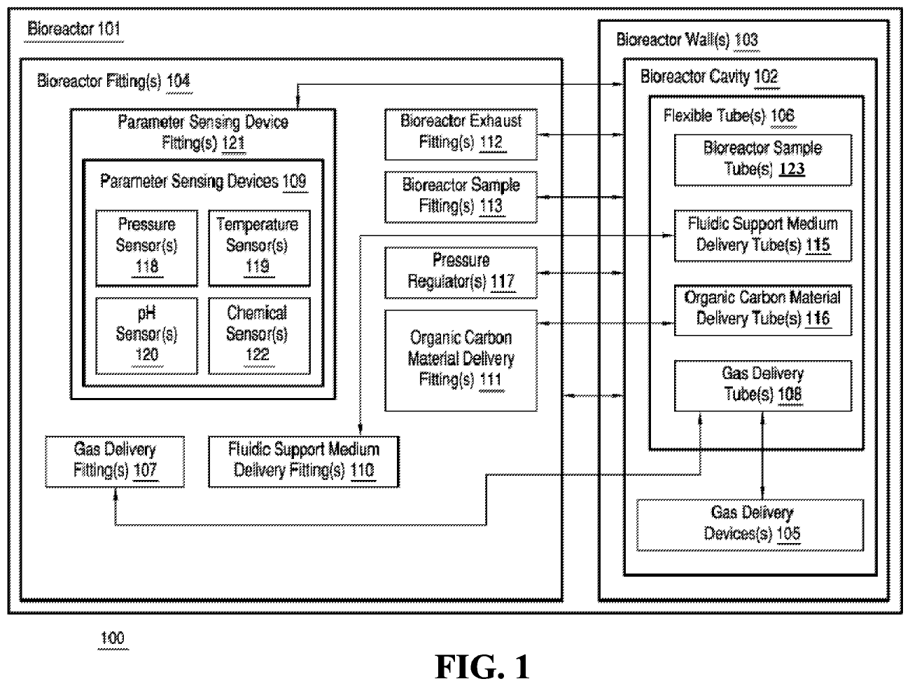

FIG. 1 illustrates an exemplary block diagram of a system 100, according to an embodiment. System 100 is merely exemplary and is not limited to the embodiments presented herein. System 100 can be employed in many different embodiments or examples not specifically depicted or described herein and such adjustments or changes can be selected by one or ordinary skill in the art without departing from the scope of the subject innovation.

System 100 comprises a bioreactor 101 that includes a bioreactor cavity 102 and one or more bioreactor walls 103. Further, bioreactor 101 can include one or more bioreactor fittings 104, one or more gas delivery devices 105, one or more flexible tubes 106, one or more parameter sensing devices 109, and/or one or more pressure regulators 117.

In many embodiments, bioreactor fitting(s) 104 can include one or more gas delivery fittings 107, one or more fluidic support medium delivery fittings 110, one or more organic carbon material delivery fittings 111, one or more bioreactor exhaust fittings 112, one or more bioreactor sample fittings 113, and/or one or more parameter sensing device fittings 121. In these or other embodiments, flexible tube(s) 106 can include one or more gas delivery tubes 108, one or more organic carbon material delivery tubes 116, one or more bioreactor sample tubes 123, and/or one or more fluidic support medium delivery tubes 115. Further, in these or other embodiments, parameter sensing device(s) 109 can include one or more pressure sensors 118, one or more temperature sensors 119, one or more pH sensors 120, and/or one or more chemical sensors 122.

Bioreactor 101 is operable to vitally support (e.g., sustain, grow, nurture, cultivate, among others) one or more organisms (e.g., one or more macroorganisms, one or more microorganisms, and the like). In these or other embodiments, the organism(s) can include one or more autotrophic organisms or one or more heterotrophic organisms. In further embodiments, the organism(s) can comprise one or more mixotrophic organisms. In many embodiments, the organism(s) can comprise one or more phototrophic organisms. In still other embodiments, the organism(s) can comprise one or more genetically modified organisms. In some embodiments, the organism(s) vitally supported by bioreactor 101 can comprise one or more organism(s) of a single type, multiple single organisms of different types, or multiple ones of one or more organisms of different types.

In many embodiments, exemplary microorganism (s) that bioreactor 101 may be implemented to vitally support can include algae (e.g., microalgae), fungi (e.g., mold), and/or cyanobacteria. For example, in many embodiments, bioreactor 101 can be implemented to vitally support multiple types of microalgae such as, but not limited to, microalgae in the classes: Eustigmatophyceae, Chlorophyceae, Prasinophyceae, Haptophyceae, Cyanidiophyceae, Prymnesiophyceae, Porphyridiophyceae, Labyrinthulomycetes, Trebouxiophyceae, Bacillariophyceae, and Cyanophyceae. The class Cyanidiophyceae includes species of Galdieria. The class Chlorophyceae includes species of Chlorella, Haematococcus, Scenedesmus, Chlamydomonas, and Micractinium. The class Prymnesiophyceae includes species of Isochrysis and Pavlova. The class Eustigmatophyceae includes species of Nannochloropsis. The class Porphyridiophyceae includes species of Porphyridium. The class Labyrinthulomycetes includes species of Schizochytrium and Aurantiochytrium. The class Prasinophyceae includes species of Tetraselmis. The class Trebouxiophyceae includes species of Chlorella. The class Bacillariophyceae includes species of Phaeodactylum. The class Cyanophyceae includes species of Spirulina. Further still, in many embodiments, bioreactor 101 can be implemented to vitally support microalgae genus and species as described here.

Bioreactor cavity 102 can hold (e.g., contain or store) the organism(s) being vitally supported by bioreactor 101, and in many embodiments, also can contain a fluidic support medium configured to hold, and in many embodiments, submerge the organism(s). In many embodiments, the fluidic support medium can comprise a culture medium, and the culture medium can comprise, for example, water. The bioreactor cavity 102 can be at least partially formed and enclosed by one or more bioreactor wall(s) 103. When the bioreactor 101 is implemented with bioreactor fitting(s) 104, bioreactor fitting(s) 104 together with bioreactor wall(s) 103 can fully form and enclose bioreactor cavity 102. Further, as explained in greater detail below, bioreactor wall(s) 103 and one or more of bioreactor fitting(s) 104, as applicable, can be operable to at least partially (e.g., fully) seal the contents of bioreactor cavity 102 (e.g., the organism(s) and/or fluidic support medium) within bioreactor cavity 102. As a result, the bioreactor 101 can maintain conditions mitigating the risk of introducing foreign (e.g., unintended) and/or contaminating organisms to bioreactor cavity 102. In other words, bioreactor 101 can engender the dominance (e.g., proliferation) of certain (e.g., intended) organism(s) being vitally supported at bioreactor 102 over foreign (e g, unintended) and/or contaminating organisms. For example, bioreactor 101 can maintain substantially (e.g., absolutely) axenic conditions in the bioreactor cavity 102.

Bioreactor wall(s) 103 comprise one or more bioreactor wall materials. When bioreactor wall(s) 103 comprise multiple bioreactor walls, two or more of the bioreactor walls can comprise the same bioreactor wall material(s) and/or two or more of the bioreactor walls can comprise different bioreactor wall material(s). In many embodiments, part or all of the bioreactor wall material(s) can comprise (e.g., consist of) one or more flexible materials. In some embodiments, bioreactor 101 can comprise a bag bioreactor.

In these or other embodiments, part or all of the bioreactor wall material(s) (e.g., the flexible material(s)) can comprise one or more partially transparent (e.g., fully transparent) and/or partially translucent (e.g., fully translucent) materials, such as, for example, when bioreactor 101 comprises a photobioreactor (e.g., when the organism(s) comprise phototrophic organism(s)). For example, implementing the bioreactor wall material(s) (e.g., the flexible material(s)) with at least partially transparent or translucent materials can permit light radiation to pass through bioreactor wall(s) 103 to be used as an energy source by the organism(s) contained at bioreactor cavity 102. Still, in some embodiments, bioreactor 101 can vitally support phototrophic organisms when the bioreactor wall material(s) (e.g., the flexible material(s)) of bioreactor wall(s) 103 are opaque, such as, for example, by providing sources of light radiation internal to bioreactor cavity 102. Further, in some embodiments, part or all of the bioreactor wall material(s) (e.g., the flexible material(s)) can comprise one or more selectively partially transparent (e.g., fully transparent) and/or partially translucent (e.g., fully translucent) materials, able to shift from opaque to at least partial transparency (e.g., full transparency) or at least partial translucency (e.g., full translucency).

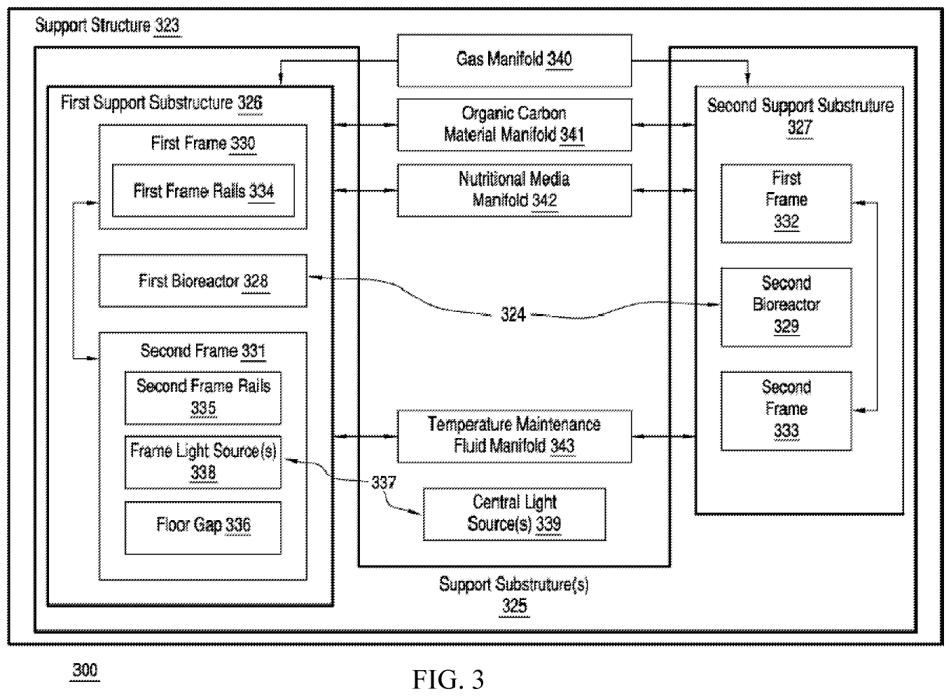

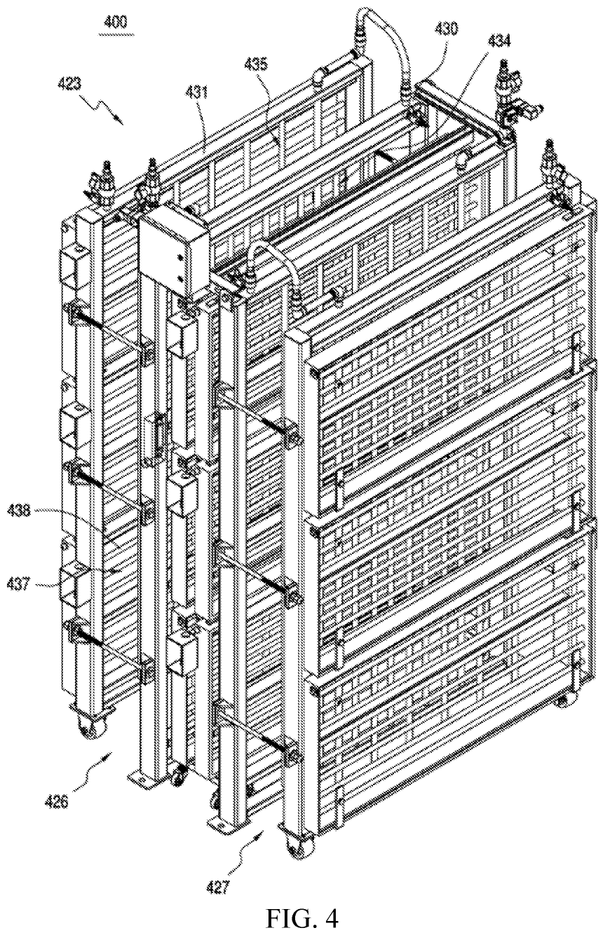

Bioreactor cavity 102 can comprise a cavity volume. The cavity volume of bioreactor cavity 102 can comprise any desirable volume. However, in some embodiments, the cavity volume can be constrained by an available geometry (e.g., the dimensions) of the sheet material(s) used to manufacture bioreactor wall(s) 103. Other factors that can constrain the cavity volume can include a light penetration depth through bioreactor wall(s) 103 and into bioreactor cavity 102 (e.g., when the organism(s) vitally supported by bioreactor 101 are phototrophic organism(s)), a size of an available autoclave for sterilizing bioreactor 101, and/or a size of a support structure implemented to mechanically support bioreactor 101. For example, the support structure can be similar or identical to support structure 323 (shown in FIG. 3) and/or support structure 423 (as shown in FIG. 4).

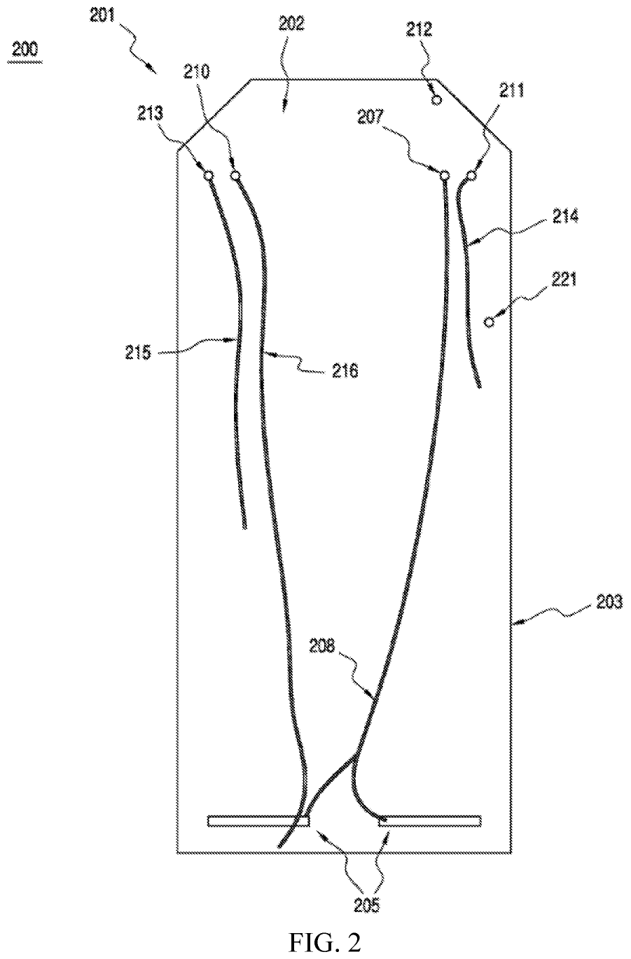

FIG. 2 illustrates a schematic side view of a system 200, according to an embodiment. System 200 is a non-limiting example of system 100 (as shown in FIG. 1). Yet, system 200 of FIG. 2 can be modified or substantially similar to the system 100 of FIG. 1 and such modifications can be selected by one or ordinary skill in the art without departing from the scope of this innovation.

System 200 can comprise bioreactor 201, bioreactor cavity 202, one or more bioreactor walls 203, one or more gas delivery devices 205, one or more gas delivery fittings 207, one or more gas delivery tubes 208, one or more fluidic support medium delivery fittings 210, one or more organic carbon material delivery fittings 211, one or more bioreactor exhaust fittings 212, one or more bioreactor sample fittings 213, one or more organic carbon material delivery tubes 214, one or more bioreactor sample tubes 215, one or more fluidic support medium delivery tubes 216, and one or more parameter sensing device fittings 221. In some embodiments, bioreactor 201 can be similar or identical to bioreactor 101 (as shown in FIG. 1); bioreactor cavity 202 can be similar or identical to bioreactor cavity 102 (as shown in FIG. 1); bioreactor wall(s) 203 can be similar or identical to biore-actor wall(s) 103 (as shown in FIG. 1); gas delivery device(s) 205 can be similar or identical to gas delivery device(s) 105 (as shown in FIG. 1); gas delivery fitting(s) 207 can be similar or identical to gas delivery fitting(s) 107 (as shown in FIG. 1); gas delivery tube(s) 208 can be similar or identical to gas delivery tube(s) 108 (as shown in FIG. 1); fluidic support medium delivery fitting(s) 210 can be similar or identical to fluidic support medium delivery fitting(s) 110 (as shown in FIG. 1); organic carbon material delivery fitting(s) 211 can be similar or identical to organic carbon material delivery fitting(s) 111 (as shown in FIG. 1); bioreactor exhaust fitting(s) 212 can be similar or identical to bioreactor exhaust fitting(s) 112 (as shown in FIG. 1); bioreactor sample fitting(s) 213 can be similar or identical to bioreactor sample fitting(s) 113 (as shown in FIG. 1); organic carbon material delivery tube(s) 214 can be similar or identical to organic carbon material delivery tube(s) 116 (as shown in FIG. 1); bioreactor sample tube(s) 215 can be similar or identical to bioreactor sample tube(s) 123 (as shown in FIG. 1); fluidic support medium delivery tube(s) 216 can be similar or identical to fluidic support medium delivery tube(s) 115 (as shown in FIG. 1); and/or parameter sensing device fitting(s) 221 can be similar or identical to parameter sensing device fitting(s) 121 (as shown in FIG. 1).

Turning ahead now in the drawings, FIG. 3 illustrates an exemplary block diagram of a system 300, according to an embodiment. System 300 is merely exemplary and is not limited to the embodiments presented herein. System 300 can be employed in many different embodiments or examples not specifically depicted or described herein.

System 300 comprises a support structure 323. As explained in greater detail below, support structure 323 is operable to mechanically support one or more bioreactors 324. In these or other embodiments, as also explained in greater detail below, support structure 323 can be operable to maintain a set point temperature of one or more of bioreactor(s) 324. In many embodiments, one or more of bioreactor(s) 324 can be similar or identical to bioreactor 101 (as shown in FIG. 1) and/or bioreactor 201 (as shown in FIG. 2). Accordingly, the term set point temperature can refer to the set point temperature as defined above with respect to system 100 (as shown in FIG. 1). Further, when bioreactor(s) 324 comprise multiple bioreactors, two or more of bioreactor(s) 324 can be similar or identical to each other and/or two or more of bioreactor(s) 324 can be different form each other. For example, the bioreactor wall materials of the bioreactor walls of two or more of bioreactor(s) 324 can be different. In some embodiments, system 300 can comprise one or more of bioreactor(s) 324.

In many embodiments, support structure 323 comprises one or more support substructures 325. Each support substructure of support substructure(s) 325 can mechanically support one bioreactor or more bioreactor(s) 324. In these or other embodiments, each support substructure of support substructure(s) 325 can maintain a set point temperature of one bioreactor of bioreactor(s) 324. In further embodiments, each of support substructure(s) 325 can be similar or identical to each other.

For example, support substructure(s) 325 can comprise a first support substructure 326 and a second support substructure 327. In these embodiments, first support substructure 326 can mechanically support a first bioreactor 328 of bioreactor(s) 324, and second support substructure 327 can mechanically support a second bioreactor 329 of bioreactor(s) 324. Further, first support substructure 326 can comprise a first frame 330 and a second frame 331, and second support substructure 327 can comprise a first frame 332 and a second frame 333. In many embodiments, first frame 330 can be similar or identical to first frame 332, and second frame 331 can be similar or identical to second frame 333. Further, first frame 330 can be similar to second frame 331, and first frame 332 can be similar to second frame 333. It is to be appreciated that the first support substructure 326 can include one or more frames of a first material and the second support substructure 327 can include one or more frames of a second material.

As indicated above, first support substructure 326 can be similar or identical to second support substructure 327. Accordingly, to increase the clarity of the description of system 300 generally, the description of second support substructure 327 is limited so as not to be redundant with respect to first support substructure 326.

In many embodiments, first frame 330 and second frame 331 together can mechanically support first bioreactor 328 in interposition between first frame 330 and second frame 331. That is, bioreactor 328 can be sandwiched between first frame 330 and second frame 331 at a slot formed between first frame 330 and second frame 331. In these or other embodiments, first frame 330 and second frame 331 together can mechanically support first bioreactor 328 in an approximately vertical orientation. Further, first frame 330 and second frame 331 can be oriented approximately parallel to each other. In another embodiment, the first frame 330 and the second frame 331 can be perpendicular to one another.

In many embodiments, second frame 331 can be selectively moveable relative to first frame 330 so that the volume of the slot formed between first frame 330 and second frame 331 can be adjusted. For example, second frame 331 can be supported by one or more wheels permitting second frame 331 to be rolled closer to or further from first frame 330. Meanwhile, in these or other embodiments, second frame 331 can be coupled to first frame 330 by one or more adjustable coupling mechanisms. The adjustable coupling mechanism(s) can hold second frame 331 in a desired position relative to first frame 330 while being adjustable so that the position can be changed when desirable. In implementation, the adjustable coupling mechanism (s) can comprise one or more threaded screws extending between first frame 330 and second frame 331, such as, for example, in a direction orthogonal to first frame 330 and second frame 331. Turning the threaded screws can cause second frame 331 to move (e.g., on the wheel(s)) relative to first frame 330.

Meanwhile, in some embodiments, first frame 330 can be operable to maintain a set point temperature of first bioreactor 328 when first bioreactor 328 is operating to vitally support one or more organisms and when support structure 300 (e.g., first support substructure 326, first frame 330, and/or second frame 331) is mechanically supporting first bioreactor 328. In these or other embodiments, second frame 331 can be operable to maintain the set point temperature of first bioreactor 328 when first bioreactor 328 is operating to vitally support the organism(s) and when support structure 300 (e.g., second support substructure 327, first frame 330, and/or second frame 331) is mechanically supporting first bioreactor 328.

As indicated above, in many embodiments, second frame 331 can be similar or identical to first frame 330. Accordingly, second frame 331 can comprise multiple second frame rails 335. Meanwhile, second frame rails 335 can be similar or identical to first frame rails 334. In some embodiments, the hollow conduits of first frame rails 334 can be coupled to hollow conduits of 335. In these embodiments, the hollow conduits of first frame rails 334 and second frame rails 335 can receive the temperature maintenance fluid from the same source. However, in these or other embodiments, the hollow conduits of first frame rails 334 and the hollow conduits of second frame rails 335 can receive the temperature maintenance fluid from different sources.

In many embodiments, first support substructure 326 comprises a floor gap 336. Floor gap 336 can be located underneath one of first frame 330 or second frame 331. Floor gap 336 can permit first bioreactor 328 to bulge into floor gap 336 past first support substructure 326 when first support substructure 326 is mechanically supporting first bioreactor 328. Permitting first bioreactor 328 to bulge into floor gap 336 can relieve stress from first bioreactor 328. For example, in many embodiments, bioreactor(s) 324 can experience the greatest amount of stress at their base(s) when being mechanically supported in a vertical position, such as, for example, by support structure 323. In these embodi-ments, permitting first bioreactor 328 to bulge into floor gap 336 such that first support substructure 326 is not restraining first bioreactor 328 at floor gap 336 can relieve more stress from first bioreactor 328 than constraining all of first bioreactor 328 at both sides with first frame 330 and second frame 331, even if first frame 330 and second frame 331 are reinforced.

System 300 (e.g., support structure 323) can comprise one or more light sources 337. Light source(s) 337 can be operable to illuminate the organism(s) being vitally supported at bioreactor(s) 324. In many embodiments, second frame 331 can comprise and/or mechanically support one or more frame light source(s) 338 of light source(s) 337. Meanwhile, system 300 (e.g., support structure 323) can comprise one or more central light source(s) 339. In these or other embodiments, support substructure(s) 325 (e.g., first support substructure 326 and second support substructure 327) can be mirrored about a central vertical plane of support structure 323. Accordingly, central light source(s) 339 can be interpositioned between first support substructure 326 and second support substructure 327 so that first bioreactor 328 and second bioreactor 329 each can receive light from central light source(s) 339.

In implementation, light source(s) 337 (e.g., frame light source(s) 338 and/or central light source(s) 339) can comprise one or more banks of light bulbs and/or light emitting diodes. In some embodiments, light source(s) 337 (e.g., the light bulbs and/or light emitting diodes) can emit one or more wavelengths of light, as desirable for the particular organism(s) being vitally supported by bioreactor(s) 324.

In some embodiments, the one or more light sources 337 may be provided on one side of the bioreactors 324, and a second side of the bioreactors 324 may have no lighting devices or may have the panels with light sources pivoted open. In one non-limiting exemplary embodiment, a system 300 can include light sources 337 on a first side and an open second side to gather natural light.

Advantageously, because each support substructure of support substructure(s) 325 can maintain a set point temperature of different ones of bioreactor(s) 324, each of bioreactor(s) 324 can be maintained at a set point temperature independently of each other. For example, when bioreactor(s) 324 are vitally supporting different types of organism(s), bioreactor(s) 324 can comprise different set point temperatures. Nonetheless, in many embodiments, bioreactor(s) 324 can comprise the same set point temperatures.

Meanwhile, in many embodiments, system 300 can comprise gas manifold 340, organic carbon material manifold 341, nutritional media manifold 342, and/or temperature maintenance fluid manifold 343. Gas manifold 340 can be operable to provide gas to one or more gas delivery fittings of bioreactor(s) 324. The gas delivery fitting(s) can be similar or identical to gas delivery fitting(s) 107 (as shown in FIG. 1) and/or gas delivery fitting(s) 207 (as shown in FIG. 2). Further, organic carbon material manifold 341 can be operable to deliver organic carbon material to one or more organic carbon material delivery fittings of bioreactor(s) 324. The organic carbon material delivery fitting(s) can be similar or identical to organic carbon material delivery fitting(s) 111 (as shown in FIG. 1) and/or organic carbon material delivery fitting(s) 211 (as shown in FIG. 2). Further still, nutritional media manifold 342 can be operable to provide nutritional media to one or more fluidic support medium delivery fittings of bioreactor(s) 324. The fluidic support medium delivery fitting(s) can be similar or identical to fluidic support medium delivery fitting(s) 110 (as shown in FIG. 1) and/or fluidic support medium delivery fitting(s) 210 (as shown in FIG. 2). Meanwhile, temperature maintenance fluid manifold can be configured to provide the temperature maintenance fluid to the hollow conduits of first frame 330 and/or second frame 331.

Gas manifold 340, organic carbon material manifold 341, nutritional media manifold 342, and/or temperature maintenance fluid manifold 343 each can comprise one or more tubes, one or more valves, one or more gaskets, one or more reservoirs, one or more pumps, and/or control logic (e.g., one or more computer processors, one or more transitory memory storage modules, and/or one or more non-transitory memory storage modules) configured to perform their respective functions. In these embodiments, the control logic can communicate with one or more parameter sensing devices of bioreactor(s) 324 to determine when to perform their respective functions (i.e., according to the needs of the organism(s) being vitally supported by bioreactor(s) 324). The parameter sensing device(s) can be similar or identical to parameter sensing device(s) 109 (as shown in FIG. 1).

Turning to the next drawing, FIG. 4 illustrates a system 400, according to an embodiment. System 400 is a non-limiting example of system 300 (as shown in FIG. 3). Yet, system 400 of FIG. 4 can be modified or substantially similar to the system 300 of FIG. 3 and such modifications can be selected by one or ordinary skill in the art without departing from the scope of this innovation.

System 400 can comprise support structure 423, first support substructure 426, second support substructure 427, first frame 430, second frame 431, first frame rails 434, second frame rails 435, and one or more light source(s) 437. In these embodiments, light source(s) 437 can comprise one or more frame light sources 438. In many embodiments, support structure 423 can be similar or identical to support structure 323 (as shown in FIG. 3); first support substructure 426 can be similar or identical to first support substructure 326 (as shown in FIG. 3); second support substructure 427 can be similar or identical to second support substructure 327 (as shown in FIG. 3); first frame 430 can be similar or identical to first frame 330 (as shown in FIG. 3); second frame 431 can be similar or identical to second frame 331 (as shown in FIG. 3); first frame rails 434 can be similar or identical to first frame rails 334 (as shown in FIG. 3); second frame rails 435 can be similar or identical to second frame rails 335 (as shown in FIG. 3); and/or light source(s) 437 can be similar or identical to light source(s) 337 (as shown in FIG. 3). Further, frame light source(s) 438 can be similar or identical to frame light source(s) 338.

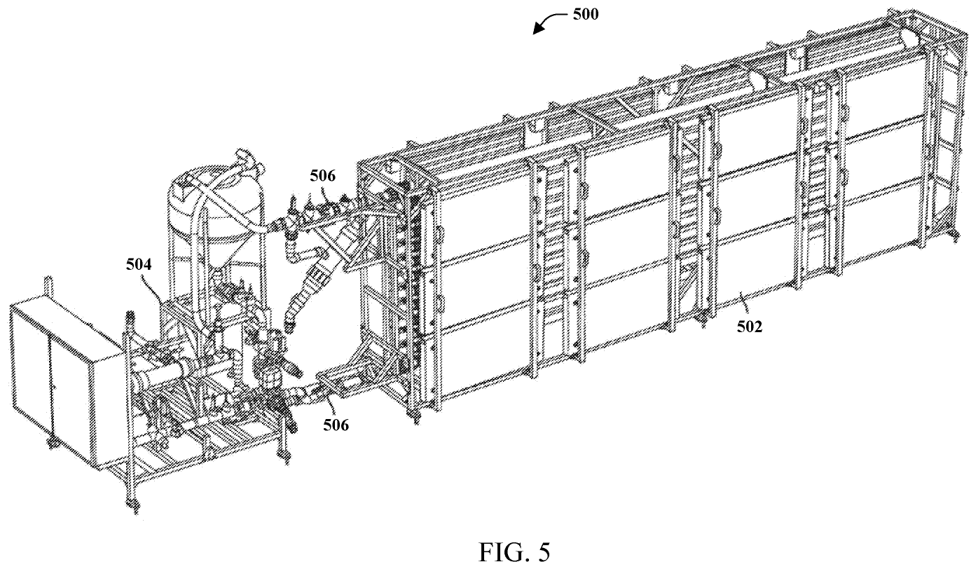

FIG. 5 illustrates an embodiment of a modular bioreactor system 500. In one embodiment, a self-contained bioreactor system for culturing microorganisms in an aqueous medium comprises a modular bioreactor system. The modular bioreactor system comprises a plurality of modular components which may be easily coupled together into a functioning system and decoupled for repair, replacement, upgrading, shipping, cleaning, or reconfiguration. The interchangeability of the modular components allows components of a bioreactor system to be easily transported and assembled at multiple locations, as well as to change the capacity of the bioreactor system or change the functionality of the bioreactor system. Each module is a standalone unit that may be interchanged with other modular bioreactor systems for different configurations, providing the benefit of flexibility over conventional single configuration integrated bioreactor systems.

In some embodiments, the modular components may be decoupled when the modular bioreactor system contains an aqueous culture of microorganisms, while maintaining isolated volumes of the aqueous microorganism culture in the various individual modular components without exposing the culture of microorganisms to the environment or outside contamination. With the ability to maintain an isolated volume of the aqueous culture, modules may be interchanged in the event of equipment malfunction without necessitating harvest or enduring a complete loss of the microorganism culture. Additionally, an isolated volume of the aqueous microorganism culture may be transported to different locations for different operations, such as growth, product maturation (e.g., lipid accumulation, pigment accumulation), harvest, dewatering, etc. The modular components may couple and decouple from each other using pipe or tubular quick connect couplers which may be quickly coupled by hand to allow fluid communication between modular components and quickly decoupled in a manner which also self-seals any fluid communication, effectively sealing an isolated volume of the aqueous culture in each modular component. The quick connect couplers may comprise fluid conduit couplers known in the art such as, but not limited to, cam lock couplers.

A non-limiting exemplary embodiment of a modular bioreactor system 500 is shown in FIG. 5. FIG. 5 shows a modular bioreactor system 500 with a bioreactor module 502, cleaning module 504, and pump and control module 506 coupled together in fluid communication. It is to be appreciated that the modular bioreactor system 500 with a bioreactor module 502, cleaning module 504, and pump and control module 506 can be decoupled from each other. As an example, one or more couplers between the modules may comprise quick connection couplers such as, but not limited to cam lock couplers, capable of self-sealing an isolated volume of an aqueous culture medium in each individual module. In some embodiments of the modular bioreactor system 500, the couplers may comprise traditional couplers such as, but not limited to, threaded connections or bolted together flange connections.

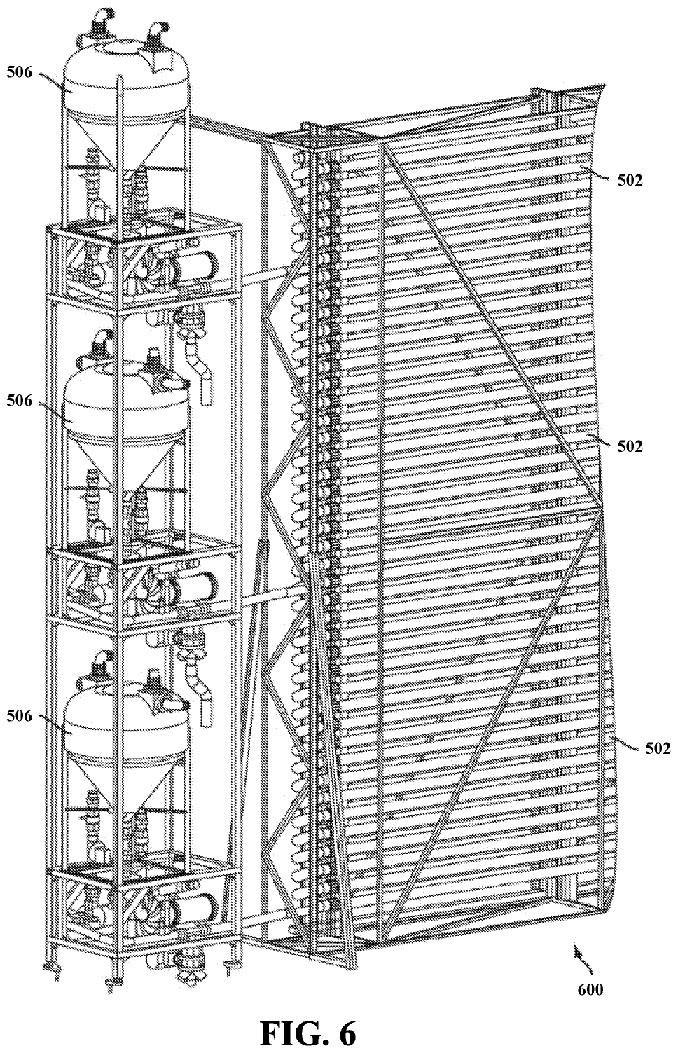

FIG. 6 illustrates a non-limiting exemplary embodiment of a cascading transfer bioreactor system 600 with multiple bioreactor modules 502 and multiple pump and control modules 506. The cascading transfer bioreactor system 600 can include modular bioreactors may be used as a production platform, as a seed reactor platform, or a combination of both. The cascading transfer bioreactor system 600 may be used in a system that connects the seed production with one or more larger volume downstream production reactors. The cascading transfer bioreactor system 600 may be partially or fully harvested to inoculate a larger seed reactor. The cascading transfer bioreactor system 600 may be used as a finishing step for the production of products that require a two-step growth process to produce pigments or other high value products.

In an alternate embodiment, the cascading transfer bioreactor system 600 may comprise culture tube segments that have different diameters, where a small diameter is used for a preferentially phototrophic section while a larger tubular diameter is used for a preferably mixotrophic section. The segments with different culture tube diameters may be interleaved and connected in a way to enhance turbulence or mixing in the system without the use of a high Reynolds numbers such that the overall system pressure drop is reduced.

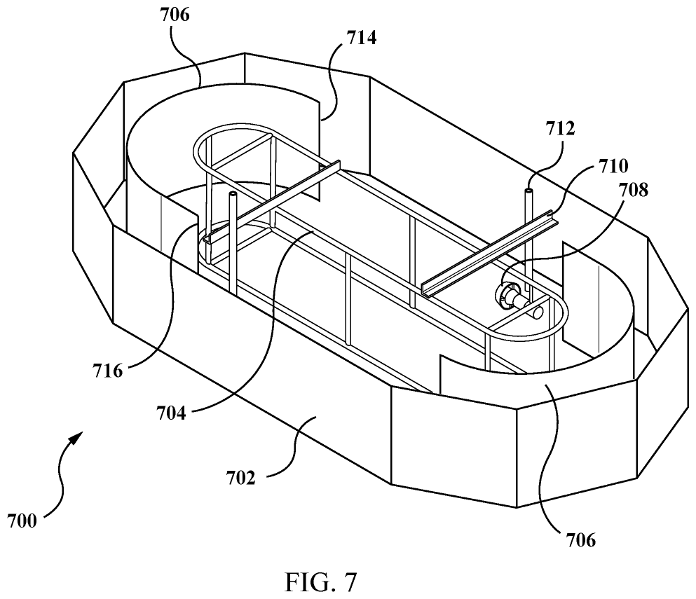

Turning to FIG. 7, a non-limiting embodiment of the open raceway pond bioreactor 700 is illustrated. The open raceway pond bioreactor 700 comprises an outer wall 702, center wall 704, arched turning vanes 706, submerged thrusters 708, support structure 710 (horizontal), and 712 (vertical). The outer wall 702 and the center wall 704 form the boundaries of the straight away portions and U-bend portions of the bioreactor 700. The center wall 704 is shown as a frame for viewing purposes, but in practice panels are inserted into open sections of the frame or a liner placed over the frame to form a solid center wall surface. Also, the outer wall 702 of the bioreactor 700 is depicted as multiple straight segments connected at angles to form the curved portion of the U-bend, but the outer wall 702 may also form a continuous curve or arc.

The arched turning vanes 706 can have an asymmetrical shape having a first end 714 of the turning vane at the beginning of the U-bend portion and a second end 716 extending past the U-bend portion into the straight away portion. The flow path of the culture in the open raceway pond bioreactor 700 would be counter clockwise, with the culture encountering first end 714 of the turning vane first, second end 716 of the turning vane second, and then the submerged thruster 708 when traveling through the U-bend portion and into the straight away portion. The arched turning vanes 706 are also shown in to be at least as tall as the center wall 704, to allow a portion of the arched turning vanes 706 to protrude from the culture volume when operating.

In a review of literature to determine the nitrogen sources commonly used in microalgae cultivation, it was noted most microalgae culture media are designed to support photosynthetic growth, as opposed to mixotrophic or heterotrophic growth. The processing of carbon dioxide by the microalgae in photosynthesis results in an alkalization of the culture media (i.e., an increase in the culture medium pH). The ammonia or ammonium toxicity of a microalgae culture increases exponentially with an increase in pH, and thus using nitrates as a nitrogen source poses a lower risk to impaired growth of the microalgae as a result of ammonia or ammonium toxicity. A review of Andersen (2005) Appendix A from: Algal Culturing Techniques. Ed. Andersen, Burlington, Mass.: Elsevier/Academic, pp 431-532, reflected the photosynthesis bias in culture media recipes and the corresponding preference for nitrates. Of the 59 culture media recipes reviewed, 80% used nitrates, 13% used a combination of ammonia and nitrates, and 7% used ammonia as the nitrogen source. Other nitrogen sources that may suitable for use with microalgae cultures may comprise monosodium glutamate.

While culturing microalgae in mixotrophic or heterotrophic culture conditions utilizing ammonium or ammonia as a nitrogen source is known, the inventors have developed methods to leverage the ammonia or ammonium toxicity level in a culture of microalgae to induce the uptake of nitrogen for controlling the metabolism of the microalgae. Contemplated benefits of these methods include, but are not limited to: increasing the metabolic rate of the microalgae, increasing the respiration rate of the microalgae, reducing the culturing time for production of specific metabolite (e.g., protein) by the microalgae; increasing the initial rate of growth in a microalgae culture, and increasing the accumulation of protein.

Non-limiting examples of suitable microalgae for mixotrophic or heterotrophic growth using acetic acid or acetate as an organic carbon source may comprise microalgae of the genera: Chlorella, Anacystis, Synechococcus, Synechocystis, Neospongiococcum, Chlorococcum, Phaeodactylum, Spirulina, Micractinium, Haematococcus, Nannochloropsis, Brachiomonas, Schizochytrium, Aurantiochytrium, Crypthecodinium, Chlamydomonas, Euglena, and species thereof. Non-limiting examples of other microalgae capable of mixotrophic or heterotrophic growth on a various organic carbon sources may comprise: Tetraselmis, Nitzschia, Galdieria, Agmenellum, Goniotrichium, Navicula, Phaeodactylum, Rhodomonas, Cyclotella, Skeletonema, Pavlova, Dunaliella, and species thereof. Organic carbon sources suitable for growing microalgae mixotrophically or heterotrophically may comprise: acetate, acetic acid, ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolyzate, proline, propionic acid, ribose, sacchrose, partial or complete hydrolysates of starch, sucrose, tartaric, TCA-cycle organic acids, thin stillage, urea, agricultural by-products, industrial process by-products, municipal waste streams, yeast extract, xylose, and combinations thereof. The organic carbon source may comprise any single source, combination of sources, and dilutions of single sources or combinations of sources.

Analysis of the DNA sequence of the strain of Chlorella sp. (HS26) described in the specification was done in the NCBI 18s rDNA reference database at the Culture Collection of Algae at the University of Cologne (CCAC) showed substantial similarity (i.e., greater than 95%) with multiple known strains of Chlorella and Micractinium. Those of skill in the art will recognize that Chlorella and Micractinium appear closely related in many taxonomic classification trees for microalgae, and strains and species may be re-classified from time to time. Thus for references throughout the instant specification for Chlorella, it is recognized that microalgae strains in related taxonomic classifications with similar characteristics to the reference Chlorella strain would reasonably be expected to produce similar results.

Additionally, taxonomic classification has also been in flux for microalgae in the genus Schizochytrium. Some organisms previously classified as Schizochytrium have been reclassified as Aurantiochytrium, Thraustochytrium, or Oblongichytrium. See Yokoyama et al. Taxonomic rearrangement of the genus Schizochytrium sensu lato based on morphology, chemotaxonomic characteristics, and 18S rRNA gene phylogeny (Thrausochytriaceae, Labyrinthulomycetes): emendation for Schizochytrium and erection of Aurantiochytrium and Oblongichytrium gen. nov. Mycoscience (2007) 48:199-211. Those of skill in the art will recognize that Schizochytrium, Aurantiochytrium, Thraustochytrium, and Oblongichytrium appear closely related in many taxonomic classification trees for microalgae, and strains and species may be re-classified from time to time. Thus for references throughout the instant specification for Schizochytrium and Aurantiochytrium, it is recognized that microalgae strains in related taxonomic classifications with similar characteristics to Schizochytrium and Aurantiochytrium would reasonably be expected to produce similar results.

An auxostat system is a type of continuous culturing system that can use feedback from sensors or other measurements taken from a culture growth location (e.g., chamber). The auxostat system uses the feedback to control various aspect of the media flow rate, and constituents, to maintain the desired culture media appropriate to the situation. The term “pH auxostat” refers to the microbial cultivation technique that couples the addition of a titrant to pH control. As the pH drifts from a given set point, the titrant is added to bring the pH back to the set point. The rate of pH change is often an excellent indication of growth and meets the requirements as a growth-dependent parameter. The titrant may keep a residual nutrient concentration (e.g., organic carbon, nitrogen) in balance with the buffering capacity of the medium. The pH set point may be changed depending on the microorganisms present in the culture at the time. The microorganisms present may be driven by the location and season where the bioreactor is operated and how close the cultures are positioned to other contamination sources (e.g., other farms, agriculture, ocean, lake, river, waste water). The rate of titrant addition is determined by the substrate consumption rate of the microorganism and the buffering capacity of the media. The pH drift of the culture is mostly driven by the nutrient consumption and therefore pH auxostat may be used to replace the nutrient that was consumed and maintaining a constant residual nutrient concentration.

In some embodiments, the inventive concepts can comprise a method that utilizes a pH auxostat to provide multiple functions comprising at least one selected from the group consisting of: supplying ammonium or ammonia to the microalgae culture as a source of nitrogen, maintaining the culture pH in a desired range, and maintaining the residual ammonia or ammonium concentration of the culture medium (i.e., ammonia or ammonium toxicity conditions) in a desired range. The toxicity of the environment is governed by a variety of factors, such as but not limited to, the total concentration of ammonia in the culture and the pH of the culture; and thus the residual ammonia or ammonium concentration of the culture medium forming the toxicity is controlled by the initial concentration of ammonia and the supply of ammonium or ammonia through the pH auxostat. Maintaining a residual ammonia or ammonium concentration in the culture medium is not inherent in a pH auxostat system, but the ability to control ammonia or ammonium toxicity in a pH auxostat system, as developed and described herein, using the described methods may produce the benefits described.

While some microalgae are known to use ammonium or ammonia as a nitrogen source, the inventors determined that an ammonia concentration that is too high can also be toxic to microalgae, and ammonia tolerance limits may vary among microalgae. Therefore, in some embodiments the developed methods operate inside a defined toxicity window that approaches the ammonia tolerance limit of the microalgae in order to control the metabolism of the microalgae, and may be achieved by deviating from the convention operation of a pH auxostat system.

In some embodiments, the exemplary method utilizes a pH auxostat to provide a supply of at least one of ammonium and ammonia to the microalgae culture as a source of nitrogen, a maintain the culture pH in a desired range, and maintain the residual ammonia or ammonium concentration of the culture medium (i.e., ammonia or ammonium toxicity conditions) in a desired range. In some embodiments, the pH auxostat system may comprise a solenoid valve, a peristaltic pump, a pH probe and a pH controller. In some embodiments, the pH auxostat system may comprise a drip application device controlled by a needle valve, a metering pump or a peristaltic pump, and a pH controller. The pH controller may be set at a threshold level (i.e., set point) and activate the auxostat system to supply at least one of ammonium and ammonia to the culture when the measured pH level is below the set threshold level. The frequency of pH measurements, administration of at least one of ammonium and ammonia by the auxostat system, and mixing of the culture are controlled in combination to keep the pH value substantially constant. In some embodiments, the at least one of ammonium and ammonia feed may be diluted in water to a concentration below 100% and as low as 0.1%, with a preferable concentration between 0.1% and 20%. In other embodiments, the at least one of ammonium and ammonia may be at concentrations below 10% in order to continuously dilute the culture of microalgae. In other embodiments, the at least one of ammonium and ammonia may be mixed together with other nutrient media, acids, bases, or organic carbon sources.

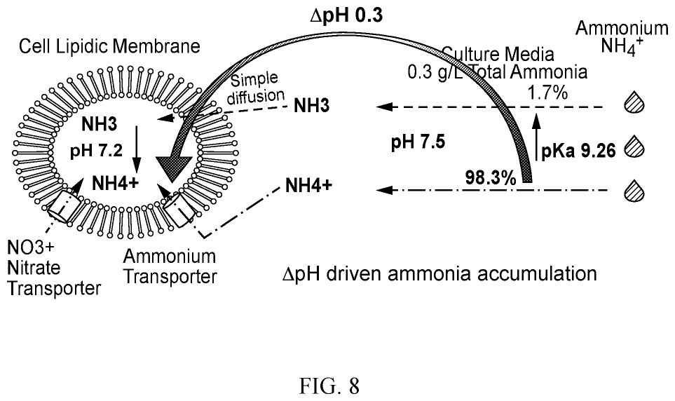

Without being bound by any particular theory, the inventors postulate that the accumulation of ammonium inside a cell is driven by the pH gradient between the internal cell pH and pH of the culture medium outside the cell. In further explanation, ammonium and free protons enter microalgae cells through an active symport transporter, while ammonia is membrane permeable and may diffuse passively into the microalgae cell. Together these characteristics allow the uptake of ammonium to be controlled by the cell, but not the diffusion of ammonia. As shown in FIG. 8, when the culture medium pH is above neutral (i.e., outside of about 7-8), the intra and extracellular dissociation equilibrium along with diffusion equilibrium of ammonia across the cell membrane results in an intracellular concentration of ammonium greater than the residual ammonium concentration in the culture medium. As further described in FIG. 8, the concentration of ammonium in a culture medium will convert to ammonia as the culture medium pH increases and approaches the pKa value of ammonia (about 9.26). The increase of available ammonia in the culture medium may increase the diffusion of ammonia through the cell membrane and into the cell with an internal pH lower than the culture medium pH. Due to the lower pH value within the cell than outside cell, at least some of the ammonia will convert into ammonium and a free proton, which increases the ammonium concentration within the cell and increase the internal cell pH. The inventors hypothesize that the concentration of ammonium in the cell may be controlled by manipulating the internal cell and culture medium pH gradient (i.e., intra/extracellular pH gradient), and that the ammonia/ammonium toxicity will be proportional to the internal cell ammonium concentration.

Thus ammonia may become toxic to microalgae when the pH gradient between the internal cell pH and pH of the culture medium outside the cell induces the buildup of ammonium inside the cells. Because the microalgae pH homeostasis will tend to maintain an internal cell pH slightly above neutral (about 7-8) in response to medium alkalization, the ammonium built up inside the cell may be modeled. The internal ammonium concentration of a cell may be calculated from the external culture pH, and the residual ammonia concentration in the culture, assuming that the internal pH of the cell and the ionic strength are maintained constant. The pH gradient between the internal cell pH and pH of the culture medium outside the cell may be calculated with the following equation derived from the Hendersen Hassleback equation, from which the internal ammonium concentration can be solved:

$$Δ pH = pHi - pHo = log \frac{( [ A ] i + [ AH ] I )}{( [ A ] O + [ AH ] 0 )}$$

pHi=pH inside the cell, pHo=pH outside the cell, AH=ammonia, A=ammonium, O=outside cell, I=inside cell. The relationship may be illustrated with the non-limiting examples in FIG. 8 showing that 1.7% of the residual ammonia concentration in the culture media may diffuse into the cell when a delta pH of 0.3 exists (culture medium pH of 7.5 and internal cell pH of 7.2). This modelling methodology may be applied generally for microalgae where the ammonia/ammonium toxicity limit is determined. As demonstrated in Table 1, the inventors determined controlling the ammonia/ammonium toxicity in a Chlorella sp. (HS26) microalgae culture may comprise maintaining a constant pH and maintaining constant residual ammonium/ammonia levels in the culture medium using the Hendersen Hassleback equation based model. Control over these parameters may aid in dictating the amount of diffusion of ammonia occurring through the microalgae cell membrane. Relevant constraints for controlling the pH may comprise, but are not limited to, the scale (e.g., size, depth, volume) of the bioreactor, the location of introduction of ammonium from a dosing system (e.g., pH auxostat), the pH control PID, amplitude, the peristaltic pump size and duty cycle for the dosing system, the aqueous culture medium buffering capacity, and the ammonium concentration in the dosing feedstock.

TABLE 1
Intracellular NH₄+/NH₃ concentration (mg/L)

| Medium NH₄+/NH₃ | Medium | Medium | Medium |
|---|---|---|---|
| concentration | pH 6.5 | pH 7.0 | pH 7.5 |
| 0.1 g/L | 0.04 | 0.35 | 3.49 |
| 0.3 g/L | 0.11 | 1.06 | 10.46 |
| 0.6 g/L | 0.21 | 2.12 | 20.92 |
| 0.9 g/L | 0.32 | 3.18 | 31.38 |
| 1.0 g/L | 0.35 | 3.53 | 34.86 |
| 1.2 g/L | 0.43 | 4.23 | 41.83 |

In some embodiments, the ammonia/ammonium toxicity threshold level of microalgae may vary based on the type of microalgae and the pH of the culture. In some embodiments, the ammonia/ammonium toxicity threshold level of Chlorella may be an intracellular concentration in the range 3-10 mg/L of NH4/NH3. In some embodiments, the ammonia/ammonium toxicity threshold level of Chlorella may be an intracellular concentration in the range 3-4 mg/L of NH4/NH3. In some embodiments, the ammonia/ammonium toxicity threshold level of Chlorella may be an intracellular concentration in the range 4-5 mg/L of NH4/NH3. In some embodiments, the ammonia/ammonium toxicity threshold level of Chlorella may be an intracellular concentration in the range 5-6 mg/L of NH4/NH3. In some embodiments, the ammonia/ammonium toxicity threshold level of Chlorella may be an intracellular concentration in the range 6-7 mg/L of NH4/NH3. In some embodiments, the ammonia/ammonium toxicity threshold level of Chlorella may be an intracellular concentration in the range 7-8 mg/L of NH4/NH3. In some embodiments, the ammonia/ammonium toxicity threshold level of Chlorella may be an intracellular concentration in the range 8-9 mg/L of NH4/NH3. In some embodiments, the ammonia/ammonium toxicity threshold level of Chlorella may be an intracellular concentration in the range 9-10 mg/L of NH4/NH3. The ammonia/ammonium toxicity of other microalgae strains such as, but not limited to, Aurantiochytrium, may be calculated and modeled in a similar fashion as that described for Chlorella and cultured in a culture pH medium ranging from 4-11.

In some embodiments, the microalgae may be cultured with a culture medium residual NH4+/NH3 concentration in the range of 0.1-2 g/L at a culture pH in the range of 6.5-7.0. In some embodiments, the microalgae may be cultured with a culture medium residual NH4+/NH3 concentration in the range of 0.1-0.3 g/L at a culture pH in the range of 6.5-7.0. In some embodiments, the microalgae may be cultured with a culture medium residual NH4+/NH3 concentration in the range of 0.3-0.5 g/L at a culture pH in the range of 6.5-7.0. In some embodiments, the microalgae may be cultured with a culture medium residual NH4+/NH3 concentration in the range of 0.5-0.7 g/L at a culture pH in the range of 6.5-7.0. In some embodiments, the microalgae may be cultured with a culture medium residual NH4+/NH3 concentration in the range of 0.7-9 g/L at a culture pH in the range of 6.5-7.0. In some embodiments, the microalgae may be cultured with a culture medium residual NH4+/NH3 concentration in the range of 0.9-1.0 g/L at a culture pH in the range of 6.5-7.0. In some embodiments, the microalgae may be cultured with a culture medium residual NH4+/NH3 concentration in the range of 1.0-1.2 g/L at a culture pH in the range of 6.5-7.0. In some embodiments, the microalgae may be cultured with a culture medium residual NH4+/NH3 concentration in the range of 1.2-1.4 g/L at a culture pH of in the range of 6.5-7.0. In some embodiments, the microalgae may be cultured with a culture medium residual NH4+/NH3 concentration in the range of 1.4-1.6 g/L at a culture pH in the range of 6.5-7.0. In some embodiments, the microalgae may be cultured with a culture medium residual NH4+/NH3 concentration in the range of 1.6-1.8 g/L at a culture pH in the range of 6.5-7.0. In some embodiments, the microalgae may be cultured with a culture medium residual NH4+/NH3 concentration in the range of 1.8-2.0 g/L at a culture pH in the range of 6.5-7.0.

In some embodiments, the microalgae may be cultured with a culture medium residual NH4+/NH3 concentration less than or equal to 2.0 g/L at a culture pH in the range of 6.5-7.0. In some embodiments, the microalgae may be cultured with a culture medium residual NH4+/NH3 concentration less than or equal to 1.8 g/L at a culture pH in the range of 6.5-7.0. In some embodiments, the microalgae may be cultured with a culture medium residual NH4+/NH3 concentration less than or equal to 1.6 g/L at a culture pH in the range of 6.5-7.0. In some embodiments, the microalgae may be cultured with a culture medium residual NH4+/NH3 concentration less than or equal to 1.4 g/L at a culture pH in the range of 6.5-7.0. In some embodiments, the microalgae may be cultured with a culture medium residual NH4+/NH3 concentration less than or equal to 1.2 g/L at a culture pH in the range of 6.5-7.0. In some embodiments, the microalgae may be cultured with a culture medium residual NH4+/NH3 concentration less than or equal to 1.0 g/L at a culture pH in the range of 6.5-7.0. In some embodiments, the microalgae may be cultured with a culture medium residual NH4+/NH3 concentration less than or equal to 0.8 g/L at a culture pH in the range of 6.5-7.0. In some embodiments, the microalgae may be cultured with a culture medium residual NH4+/NH3 concentration less than or equal to 0.6 g/L at a culture pH in the range of 6.5-7.0. In some embodiments, the microalgae may be cultured with a culture medium residual NH4+/NH3 concentration less than or equal to 0.4 g/L at a culture pH in the range of 6.5-7.0. In some embodiments, the microalgae may be cultured with a culture medium residual NH4+/NH3 concentration less than or equal to 0.2 g/L at a culture pH in the range of 6.5-7.0.

In some embodiments, the microalgae may be cultured with a culture medium residual NH4+/NH3 concentration in the range of 0.1-2 g/L at a culture pH in the range of 7.0-7.5. In some embodiments, the microalgae may be cultured with a culture medium residual NH4+/NH3 concentration in the range of 0.1-0.3 g/L at a culture pH in the range of 7.0-7.5. In some embodiments, the microalgae may be cultured with a culture medium residual NH4+/NH3 concentration in the range of 0.3-0.5 g/L at a culture pH in the range of 7.0-7.5. In some embodiments, the microalgae may be cultured with a culture medium residual NH4+/NH3 concentration in the range of 0.5-0.7 g/L at a culture pH in the range of 7.0-7.5. In some embodiments, the microalgae may be cultured with a culture medium residual NH4+/NH3 concentration in the range of 0.7-9 g/L at a culture pH in the range of 7.0-7.5. In some embodiments, the microalgae may be cultured with a culture medium residual NH4+/NH3 concentration in the range of 0.9-1.0 g/L at a culture pH in the range of 7.0-7.5. In some embodiments, the microalgae may be cultured with a culture medium residual NH4+/NH3 concentration in the range of 1.0-1.2 g/L at a culture pH in the range of 7.0-7.5. In some embodiments, the microalgae may be cultured with a culture medium residual NH4+/NH3 concentration in the range of 1.2-1.4 g/L at a culture pH of in the range of 7.0-7.5. In some embodiments, the microalgae may be cultured with a culture medium residual NH4+/NH3 concentration in the range of 1.4-1.6 g/L at a culture pH in the range of 7.0-7.5. In some embodiments, the microalgae may be cultured with a culture medium residual NH4+/NH3 concentration in the range of 1.6-1.8 g/L at a culture pH in the range of 7.0-7.5. In some embodiments, the microalgae may be cultured with a culture medium residual NH4+/NH3 concentration in the range of 1.8-2.0 g/L at a culture pH in the range of 7.0-7.5.

In some embodiments, the microalgae may be cultured with a culture medium residual NH4+/NH3 concentration less than or equal to 2.0 g/L at a culture pH in the range of 7.0-7.5. In some embodiments, the microalgae may be cultured with a culture medium residual NH4+/NH3 concentration less than or equal to 1.8 g/L at a culture pH in the range of 7.0-7.5. In some embodiments, the microalgae may be cultured with a culture medium residual NH4+/NH3 concentration less than or equal to 1.6 g/L at a culture pH in the range of 7.0-7.5. In some embodiments, the microalgae may be cultured with a culture medium residual NH4+/NH3 concentration less than or equal to 1.4 g/L at a culture pH in the range of 7.0-7.5. In some embodiments, the microalgae may be cultured with a culture medium residual NH4+/NH3 concentration less than or equal to 1.2 g/L at a culture pH in the range of 7.0-7.5. In some embodiments, the microalgae may be cultured with a culture medium residual NH4+/NH3 concentration less than or equal to 1.0 g/L at a culture pH in the range of 7.0-7.5. In some embodiments, the microalgae may be cultured with a culture medium residual NH4+/NH3 concentration less than or equal to 0.8 g/L at a culture pH in the range of 7.0-7.5. In some embodiments, the microalgae may be cultured with a culture medium residual NH4+/NH3 concentration less than or equal to 0.6 g/L at a culture pH in the range of 7.0-7.5. In some embodiments, the microalgae may be cultured with a culture medium residual NH4+/NH3 concentration less than or equal to 0.4 g/L at a culture pH in the range of 7.0-7.5. In some embodiments, the microalgae may be cultured with a culture medium residual NH4+/NH3 concentration less than or equal to 0.2 g/L at a culture pH in the range of 7.0-7.5.

In some embodiments, the microalga may be cultured with a culture medium residual NH4+/NH3 concentration in the range of 0.01-0.50 g/L at a culture pH of 7.5-8.0. In some embodiments, the microalga may be cultured with a culture medium residual NH4+/NH3 concentration in the range of 0.01-0.05 g/L at a culture pH of 7.5-8.0. In some embodiments, the microalga may be cultured with a culture medium residual NH4+/NH3 concentration in the range of 0.05-0.10 g/L at a culture pH of 7.5-8.0. In some embodiments, the microalga may be cultured with a culture medium residual NH4+/NH3 concentration in the range of 0.10-0.15 g/L at a culture pH of 7.5-8.0. In some embodiments, the microalga may be cultured with a culture medium residual NH4+/NH3 concentration in the range of 0.15-0.20 g/L at a culture pH of 7.5-8.0. In some embodiments, the microalga may be cultured with a culture medium residual NH4+/NH3 concentration in the range of 0.2.0-0.25 g/L at a culture pH of 7.5-8.0. In some embodiments, the microalga may be cultured with a culture medium residual NH4+/NH3 concentration in the range of 0.25-0.30 g/L at a culture pH of 7.5-8.0. In some embodiments, the microalga may be cultured with a culture medium residual NH4+/NH3 concentration in the range of 0.30-0.35 g/L at a culture pH of 7.5-8.0. In some embodiments, the microalga may be cultured with a culture medium residual NH4+/NH3 concentration in the range of 0.35-0.40 g/L at a culture pH of 7.5-8.0. In some embodiments, the microalga may be cultured with a culture medium residual NH4+/NH3 concentration in the range of 0.40-0.45 g/L at a culture pH of 7.5-8.0. In some embodiments, the microalga may be cultured with a culture medium residual NH4+/NH3 concentration in the range of 0.45-0.50 g/L at a culture pH of 7.5-8.0.

In some embodiments, the microalgae may be cultured with a culture medium residual NH4+/NH3 concentration less than or equal to 0.5 g/L at a culture pH in the range of 7.5-8.0. In some embodiments, the microalgae may be cultured with a culture medium residual NH4+/NH3 concentration less than or equal to 0.4 g/L at a culture pH in the range of 7.5-8.0. In some embodiments, the microalgae may be cultured with a culture medium residual NH4+/NH3 concentration less than or equal to 0.3 g/L at a culture pH in the range of 7.5-8.0. In some embodiments, the microalgae may be cultured with a culture medium residual NH4+/NH3 concentration less than or equal to 0.2 g/L at a culture pH in the range of 7.5-8.0. In some embodiments, the microalgae may be cultured with a culture medium residual NH4+/NH3 concentration less than or equal to 0.1 g/L at a culture pH in the range of 7.5-8.0.

In some embodiments, the microalga may be cultured with a culture medium residual NH4+/NH3 concentration in the range of 0.01-0.5 g/L at a culture pH of 8.0-8.5. In some embodiments, the microalga may be cultured with a culture medium residual NH4+/NH3 concentration in the range of 0.01-0.05 g/L at a culture pH of 8.0-8.5. In some embodiments, the microalga may be cultured with a culture medium residual NH4+/NH3 concentration in the range of 0.05-0.10 g/L at a culture pH of 8.0-8.5. In some embodiments, the microalga may be cultured with a culture medium residual NH4+/NH3 concentration in the range of 0.10-0.15 g/L at a culture pH of 8.0-8.5. In some embodiments, the microalga may be cultured with a culture medium residual NH4+/NH3 concentration in the range of 0.15-0.20 g/L at a culture pH of 8.0-8.5. In some embodiments, the microalga may be cultured with a culture medium residual NH4+/NH3 concentration in the range of 0.2.0-0.25 g/L at a culture pH of 8.0-8.5. In some embodiments, the microalga may be cultured with a culture medium residual NH4+/NH3 concentration in the range of 0.25-0.30 g/L at a culture pH of 8.0-8.5. In some embodiments, the microalga may be cultured with a culture medium residual NH4+/NH3 concentration in the range of 0.30-0.35 g/L at a culture pH of 8.0-8.5. In some embodiments, the microalga may be cultured with a culture medium residual NH4+/NH3 concentration in the range of 0.35-0.40 g/L at a culture pH of 8.0-8.5. In some embodiments, the microalga may be cultured with a culture medium residual NH4+/NH3 concentration in the range of 0.40-0.45 g/L at a culture pH of 8.0-8.5. In some embodiments, the microalga may be cultured with a culture medium residual NH4+/NH3 concentration in the range of 0.45-0.50 g/L at a culture pH of 8.0-8.5.

In some embodiments, the microalgae may be cultured with a culture medium residual NH4+/NH3 concentration less than or equal to 0.5 g/L at a culture pH in the range of 8.0-8.5. In some embodiments, the microalgae may be cultured with a culture medium residual NH4+/NH3 concentration less than or equal to 0.4 g/L at a culture pH in the range of 8.0-8.5. In some embodiments, the microalgae may be cultured with a culture medium residual NH4+/NH3 concentration less than or equal to 0.3 g/L at a culture pH in the range of 8.0-8.5. In some embodiments, the microalgae may be cultured with a culture medium residual NH4+/NH3 concentration less than or equal to 0.2 g/L at a culture pH in the range of 8.0-8.5. In some embodiments, the microalgae may be cultured with a culture medium residual NH4+/NH3 concentration less than or equal to 0.1 g/L at a culture pH in the range of 8.0-8.5.

In some embodiments, a method of culturing microalgae in medium or with a feedstock comprising a low cost refined or unrefined by-product stream from industrial (e.g., manufacturing; carpet, textile, pulp, or paper milling), municipal (e.g., sewage), or agricultural (e.g., feed lots, field runoff) sources may further comprise a supply of at least one of ammonia or ammonium. In some embodiments, the refined or unrefined by-product stream from industrial, municipal, or agricultural sources may comprise: ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolyzate, proline, propionic acid, ribose, sacchrose, partial or complete hydrolysates of starch, sucrose, tartaric, TCA-cycle organic acids, thin stillage, urea, yeast extract, xylose, woody biomass, lignocellulosic biomass, food waste, beverage waste, pigments, nitrates, phosphates, phosphites, and combinations thereof. In some embodiments, the ammonia toxicity of a microalgae culture comprising a refined or unrefined by-product stream from industrial, municipal, or agricultural sources may be controlled as described through the instant specification to increase the culture life of the microalgae. In some embodiments, the ammonia toxicity of a culture of microalgae comprising refined or unrefined by-product stream from industrial, municipal, or agricultural sources may be controlled in bioreactor systems that are open or closed.

In some embodiments, a method of culturing microalgae with ammonia or ammonium in which at least one of the residual ammonia or ammonium and culture medium pH may be controlled to maintain a desired range of ammonia toxicity may be used in a microalgae culture in non-axenic conditions (e.g., culture experiencing bacterial contamination). In some embodiments, a method of culturing microalgae with ammonia or ammonium in which at least one of residual ammonia or ammonium and culture medium pH may be controlled to maintain a desired range of ammonia toxicity may be used in a microalgae culture in axenic conditions.

In one non limiting embodiment, a method of managing ammonia or ammonium toxicity for the benefit of an culture of microalgae may comprise: providing a culture in phototrophic, mixotrophic, or heterotrophic conditions; supplying the culture of microalgae with a nitrogen source comprising as least one of ammonium and ammonia; measuring a pH of the culture medium and a residual ammonia or ammonium concentration in the culture medium; and controlling the pH of the culture medium and residual ammonia or ammonium concentration in the culture medium to maintain an internal microalgae cell ammonium concentration within a calculated range to increase the protein content in the microalgae. In some embodiments, ammonium hydroxide (NH4OH) may be supplied to the culture of microalgae as a supply of nitrogen and to control the pH of the culture medium.

In some embodiments, the NH4OH may be added as a titrant by a pH auxostat system. In some embodiments, the concentration of the NH4OH titrant may be in the range of 0.1-20%. In some embodiments, the concentration of the NH4OH titrant may be in the range of 0.1-1%. In some embodiments, the concentration of the NH4OH titrant may be in the range of 0.1-0.5%. In some embodiments, the concentration of the NH4OH titrant may be in the range of 0.5-1%. In some embodiments, the concentration of the NH4OH titrant is in the range of 1-5%. In some embodiments, the concentration of the NH4OH titrant may be in the range of 5-10%. In some embodiments, the concentration of the NH4OH titrant may be in the range of 10-15%. In some embodiments, the concentration of the NH4OH titrant may be in the range of 15-20%. In some embodiments, the concentration of the NH4OH titrant may be in the range of 1-10%.

In some embodiments, the step of controlling the pH of the culture medium may further comprise the addition of at least one base selected from the group consisting of sodium hydroxide (NaOH), magnesium hydroxide (Mg[OH]2), and calcium hydroxide (Ca[OH]2). In phototrophic and mixotrophic microalgae culture conditions, the method may further comprise a supply of light comprising photosynthetically active radiation (PAR). The supply of PAR may be natural or artificial light. In mixotrophic and heterotrophic culture conditions, the method may further comprise a supply of an organic carbon source.

In some embodiments, the increase in protein in the microalgae cell may be at least 1% more compared to a microalgae culture receiving a nitrogen source that does not comprise ammonium or ammonia. In some embodiments, the increase in protein in the microalgae cell may be at least 5% more compared to a microalgae culture receiving a nitrogen source that does not comprise ammonium or ammonia. In some embodiments, the increase in protein in the microalgae cell may be at least 10% more compared to a microalgae culture receiving a nitrogen source that does not comprise ammonium or ammonia. In some embodiments, the increase in protein in the microalgae cell may be at least 15% more compared to a microalgae culture receiving a nitrogen source that does not comprise ammonium or ammonia. In some embodiments, the increase in protein in the microalgae cell may be at least 20% more compared to a microalgae culture receiving a nitrogen source that does not comprise ammonium or ammonia. In some embodiments, the increase in protein in the microalgae cell may be at least 25% more compared to a microalgae culture receiving a nitrogen source that does not comprise ammonium or ammonia. In some embodiments, the increase in protein in the microalgae cell may be at least 30% more compared to a microalgae culture receiving a nitrogen source that does not comprise ammonium or ammonia.

In some embodiments, the increase in protein in the microalgae cell may be in the range of 1-30% more compared to a microalgae culture receiving a nitrogen source that does not comprise ammonium or ammonia. In some embodiments, the increase in protein in the microalgae cell may be in the range of 1-5% more compared to a microalgae culture receiving a nitrogen source that does not comprise ammonium or ammonia. In some embodiments, the increase in protein in the microalgae cell may be in the range of 5-10% more compared to a microalgae culture receiving a nitrogen source that does not comprise ammonium or ammonia. In some embodiments, the increase in protein in the microalgae cell may be in the range of 10-15% more compared to a microalgae culture receiving a nitrogen source that does not comprise ammonium or ammonia. In some embodiments, the increase in protein in the microalgae cell may be in the range of 15-20% more compared to a microalgae culture receiving a nitrogen source that does not comprise ammonium or ammonia. In some embodiments, the increase in protein in the microalgae cell may be in the range of 20-25% more compared to a microalgae culture receiving a nitrogen source that does not comprise ammonium or ammonia. In some embodiments, the increase in protein in the microalgae cell may be in the range of 25-30% more compared to a microalgae culture receiving a nitrogen source that does not comprise ammonium or ammonia.

In some embodiments, the pH of the culture medium may be controlled to maintain a pH below 9.26 (approximately the pKa value of ammonia). In some embodiments, the pH of the culture medium may be controlled to maintain a pH in the range of 6.0-9.5. In some embodiments, the pH of the culture medium may be controlled to maintain a pH in the range of 6.5-8.0. In some embodiments, the pH of the culture medium may be controlled to maintain a pH in the range of 6.0-6.5. In some embodiments, the pH of the culture medium may be controlled to maintain a pH in the range of 6.5-7.0. In some embodiments, the pH of the culture medium may be controlled to maintain a pH in the range of 7.0-7.5. In some embodiments, the pH of the culture medium may be controlled to maintain a pH in the range of 7.5-8.0. In some embodiments, the pH of the culture medium may be controlled to maintain a pH in the range of 8.0-8.5. In some embodiments, the pH of the culture medium may be controlled to maintain a pH in the range of 8.5-9.0. In some embodiments, the pH of the culture medium may be controlled to maintain a pH in the range of 9.0-9.5.

### EXAMPLES

Embodiments of the inventive concepts described herein are exemplified and additional embodiments are disclosed in further detail in the following Examples, which are not in any way intended to limit the scope of any aspect of the techniques and systems described herein.

### Example 1

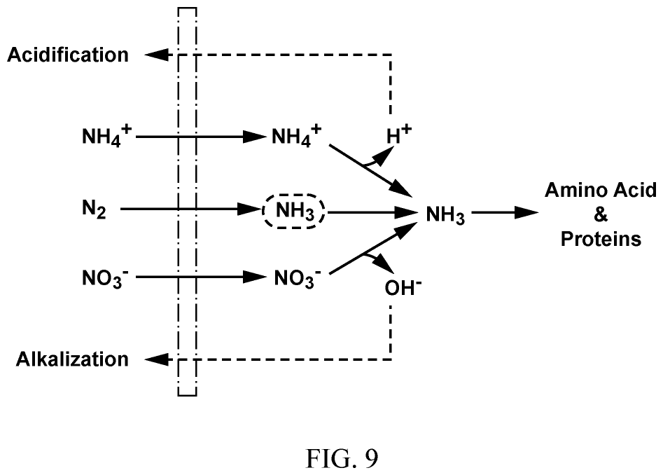

A bioreactor (e.g., one or more of the reactors discussed in FIGS. 1-7) was designed to operate as an ammonia auxostat to control both culture medium pH and residual ammonia concentration. As shown in FIG. 9, uptake and assimilation of nitrates can result in alkalization, while uptake and assimilation of ammonia can result in acidification.

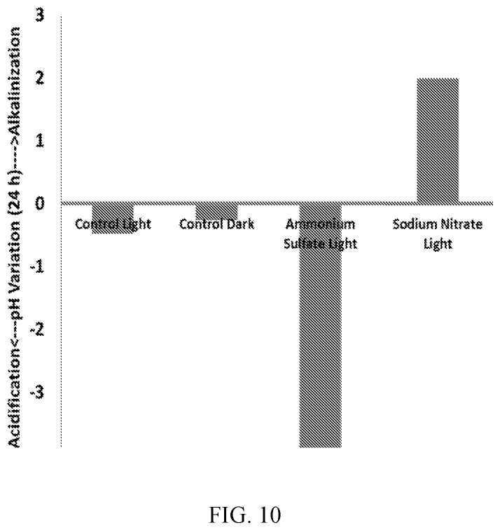

To demonstrate this different effect on pH of the different nitrogen sources, Chlorella (HS26) cells were washed with deionized water and suspended in a solution of either ammonium sulfate or sodium nitrate in flasks. The Chlorella cultures were supplied with light, and shaking from a shaker table at 100 RPM. The pH drift of each culture was measured after 24 hours and compared to a control. A shown in FIG. 10, the treatment receiving sodium nitrate increased in culture pH (i.e., alkalization), while the treatment receiving ammonium sulfate decreased in culture pH (i.e., acidification).

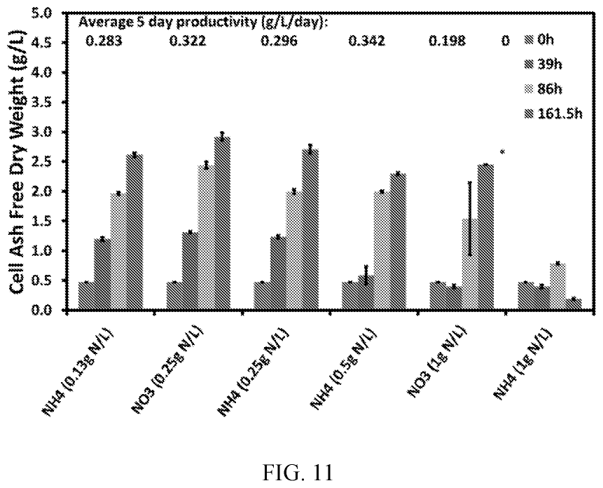

The Chlorella cultures productivity using either ammonium (NH4) or nitrates (NO3) as the nitrogen source in mixotrophic culture conditions (i.e., supply of light, supply of acetic acid as the organic carbon source) were then compared. The cultures received NH4 at a concentration of 0.13 g N/L, 0.25 g N/L, 0.5 g N/L, or 1 g N/L for the ammonium treatments. The cultures received NO3 at a concentration of 0.25 g N/L or 1 g N/L for the nitrate treatments. Samples were taken to measure cell dry weight at 0, 39, 86, and 161.5 hours. The results in FIG. 11 show that the growth and productivity of the Chlorella suffered at a concentration of 1 g N/L of NH4, and thus the ammonia toxicity of the culture must be controlled below this level. Designing an ammonia auxostat system for a bioreactor must therefore control both the residual ammonia concentration as well as the pH drift.

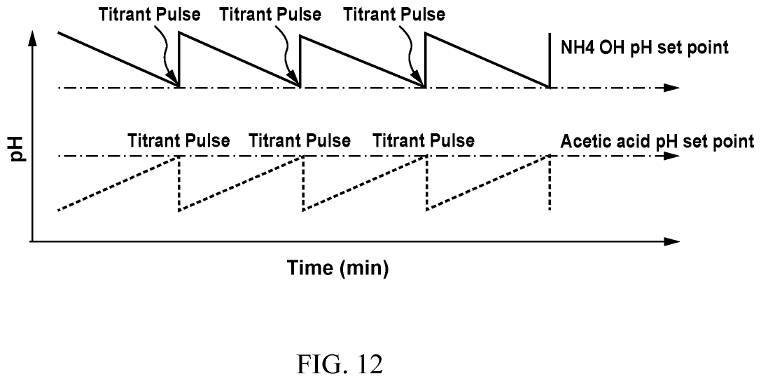

A bioreactor utilizing an acetic acid pH auxostat in mixotrophic or heterotrophic conditions typically is set up to administer acetic acid to the microalgae culture when the pH drifts above a set point (i.e., alkalization) to lower the culture pH. As shown in FIG. 12, for ammonia auxostat operation, the titrant is changed from acetic acid to ammonia hydroxide and the system administers the titrant when the pH drifts below a set point (i.e., acidification) to raise the culture pH and maintain a desired residual ammonia concentration.

### Example 2

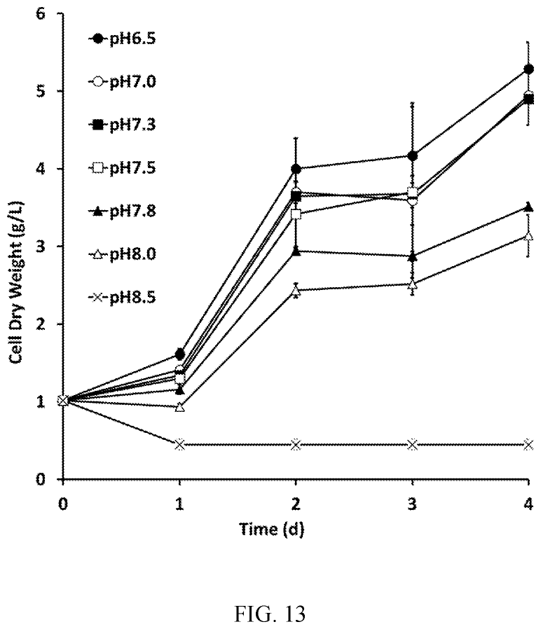

An experiment was performed to determine the effect on growth and protein accumulation of protein in a range of culture medium pH levels approaching the pKa value of ammonia (about 9.26). Cultures of Chlorella (HS26) were inoculated at 0.3 g/L in glass column bioreactors at a volume of 700 mL of BG-11 culture media and maintained at a temperature of 27° C. The mixotrophic cultures received an initial batch of 1 g/L NH4Cl, aeration at a rate of 1 Liter per minute, an initial batch of 40 g glucose/L, and were supplied 270 micromoles of light using LED lights (LumiGrow, Inc., Emeryville, Calif.). Treatments were conducted at culture medium pH values of 6.5, 7.0, 7.3, 7.5, 7.8, 8.0, and 8.5. The culture medium pH was controlled with a pH auxostat supplying a titrate of 0.5% NH4OH and 0.75% HCl at the designated set points. Samples were taken daily to measure the cell dry weight, nitrogen concentration, and total protein. Results are show in FIGS. 13-17.

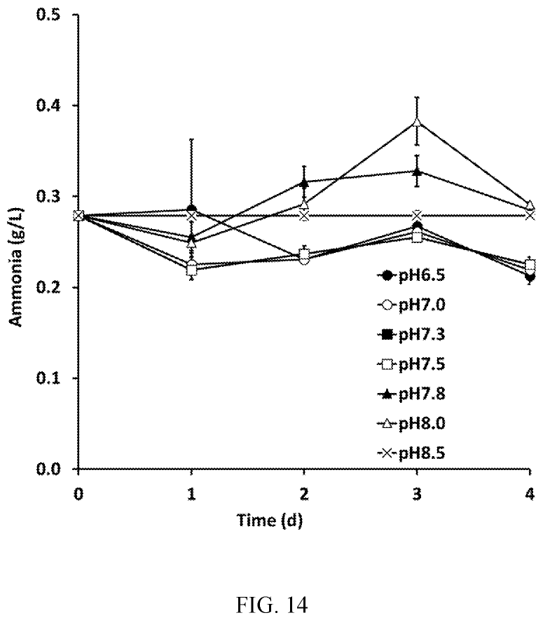

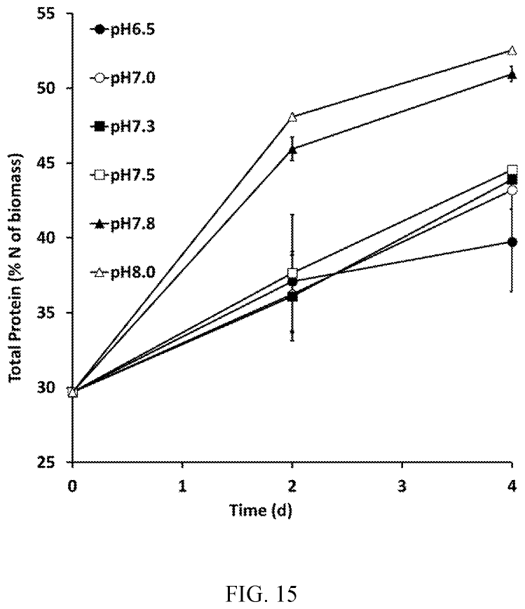

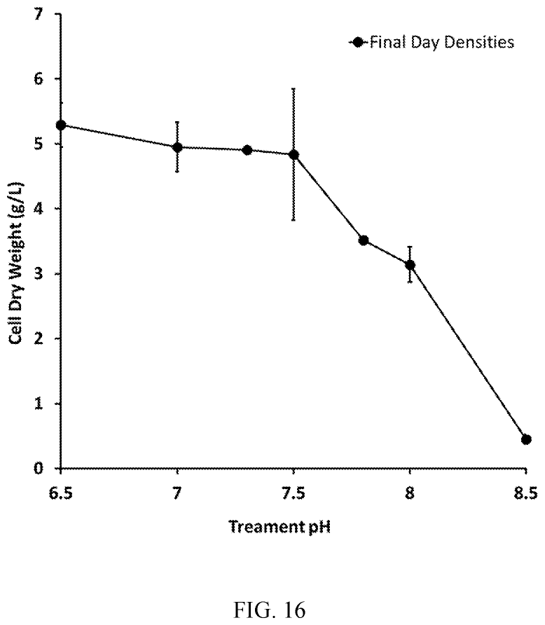

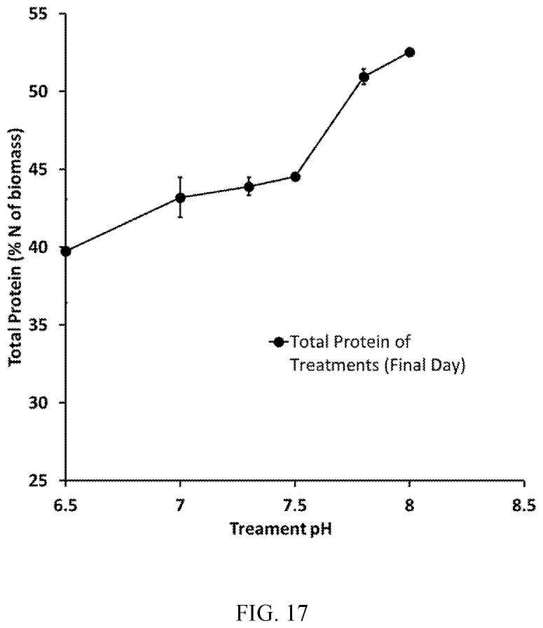

As shown in FIG. 13, all cultures grown at a culture pH of 7.5 and below grew with the standard deviation of each member of the group. All cultures grown at a culture pH above 7.5 showed signs of impaired growth due to ammonia toxicity. As shown in FIG. 14, all cultures had sufficient nitrogen for growth, and the residual ammonia concentration was held constant at about 0.25 g/L. As shown in FIG. 15, the cultures grown at a culture pH above 7.5 showed an increase in accumulated protein. As shown in FIG. 16, the final day (day 4) cell dry weight was substantially similar for the cultures growing at a pH of 7.5 and below, and then decreased as the culture pH increased. As shown in FIG. 17, the total protein on the final day (day 4) increased as the culture pH increased, particularly as the culture pH increased above 7.5. These results demonstrate that controlling the ammonia toxicity level in a culture through pH manipulation can determine culture growth conditions for microalgae that result in an increase in protein accumulation.

### Example 3

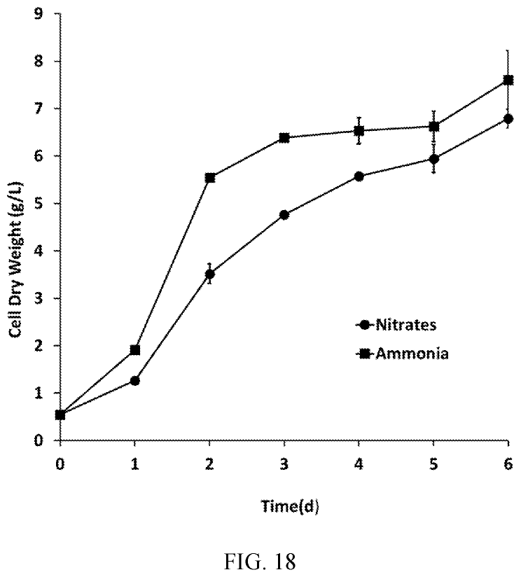

An experiment was performed to demonstrate that ammonia uptake may be induced in mixotrophic microalgae cells using an ammonium-pH auxostat system to increase growth and protein accumulation. Cultures of Chlorella (HS26) were inoculated at 0.3 g/L in glass column bioreactors at a volume of 700 mL of BG-11 culture media and maintained at a temperature of 27° C. The mixotrophic cultures received aeration at a rate of 1 Liter per minute, an initial batch of 30 g glucose/L, and were supplied 270 micromoles of light using LED lights (LumiGrow, Inc., Emeryville, Calif.). In a first treatment, the culture was supplied with an initial batch of 3 g/L NaNO3 (“Nitrate” treatment), and the culture pH was controlled at a set point of 7.0 by a pH auxostat feed containing 0.75% HCl. In a second treatment, the culture was supplied with an initial batch of 1.0 g/L NH4Cl, and the culture pH was controlled at a set point of 7.0 by a pH auxostat feed containing 0.5% NH4OH (“Ammonia” treatment). Samples were taken daily to measure the cell dry weight, nitrogen concentration, and total protein. Results are shown in FIGS. 18-21.

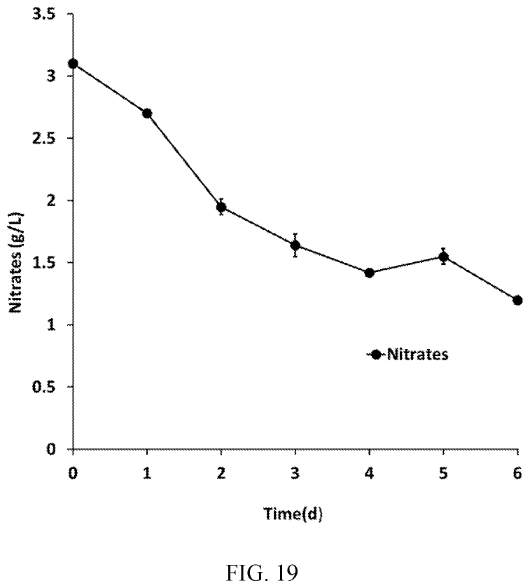

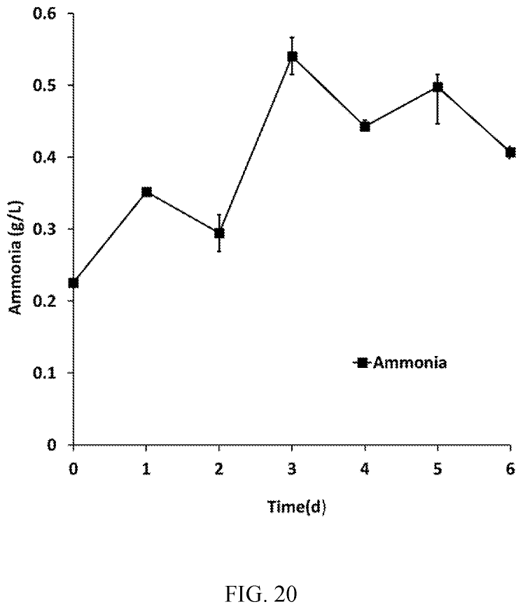

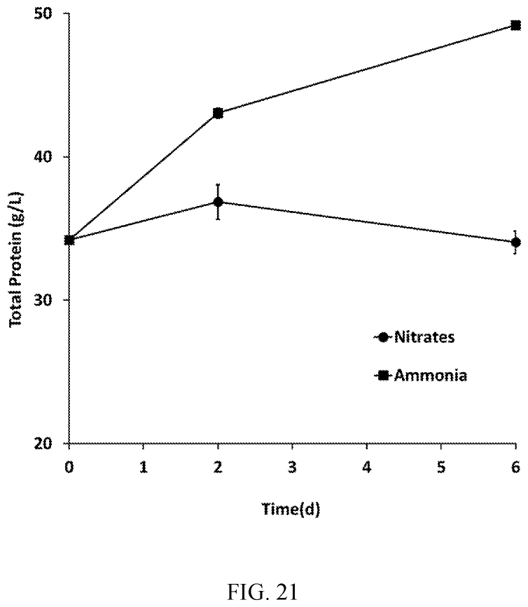

As shown in the FIG. 18, the Ammonia treatment had better growth than the Nitrate treatment. As shown in FIGS. 19-20, the cultures did not deplete all of the available nitrogen and the Ammonia treatment held the ammonia constant around 0.4 g/L. As shown in FIG. 21, the Ammonia treatment resulted in a 15% increase in protein. Therefore, the results illustrate that utilizing the Ammonia treatment, comprising an ammonium-pH auxostat system, microalgae growth rate and protein accumulation were able to be increased when compared to the Nitrate treatment.

### Example 4

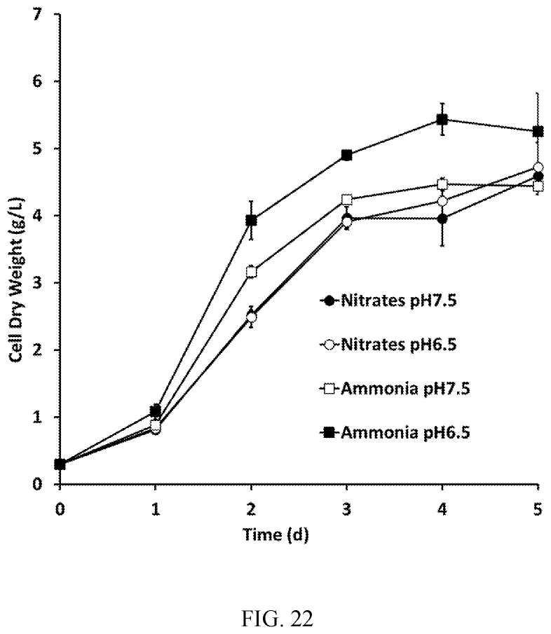

A demonstration was undertaken to show that ammonia uptake may be induced in mixotrophic microalgae cells using an ammonium-pH auxostat system to increase growth and protein accumulation. Cultures of Chlorella (HS26) were inoculated at 0.3 g/L in glass column bioreactors at a volume of 700 mL of BG-11 culture media and maintained at a temperature of 27° C. The mixotrophic cultures received aeration at a rate of 1 Liter per minute, an initial batch of 30 g glucose/L, and were supplied 270 micromoles of light using LED lights (LumiGrow, Inc., Emeryville, Calif.). In a first treatment, the culture was supplied with an initial batch of 3 g/L NaNO3 (“Nitrate” treatment), and the culture pH was controlled at a set point of 6.5 by a pH auxostat feed containing 0.50% HCl. In a second treatment, the culture was supplied with an initial batch of 3 g/L NaNO3 (“Nitrates” treatment), and the culture pH was controlled at a set point of 7.5 by a pH auxostat feed containing 0.50% HCl. In a third treatment, the culture was supplied with an initial batch of 1.0 g/L NH4Cl, and the culture pH was controlled at a set point of 6.5 by a pH auxostat feed containing 0.25% NH4OH (“Ammonia” treatment). In a fourth treatment, the culture was supplied with an initial batch of 1.0 g/L NH4Cl, and the culture pH was controlled at a set point of 7.5 by a pH auxostat feed containing 0.25% NH4OH (“Ammonia” treatment). Samples were taken daily to measure the cell dry weight, nitrogen concentration, and total protein. Results are shown in FIGS. 22-25.

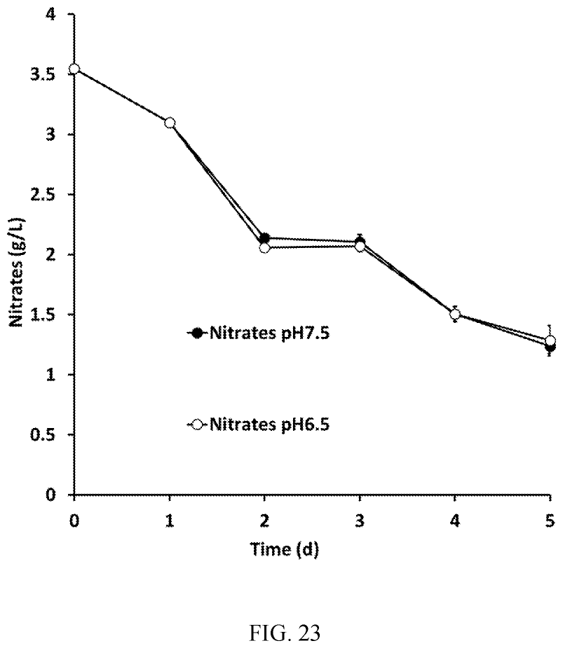

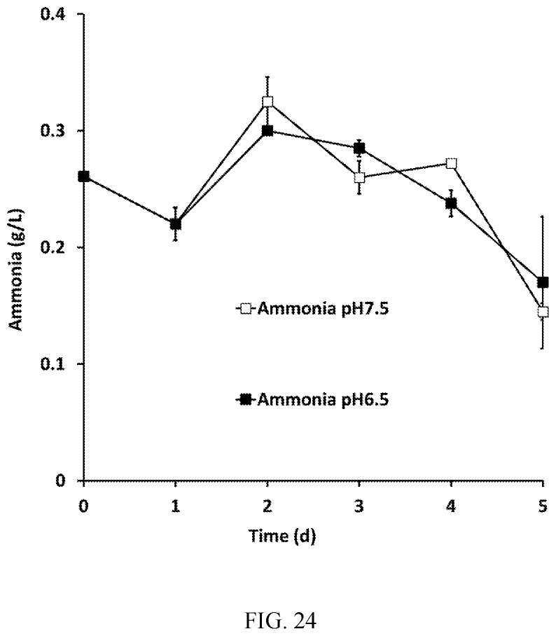

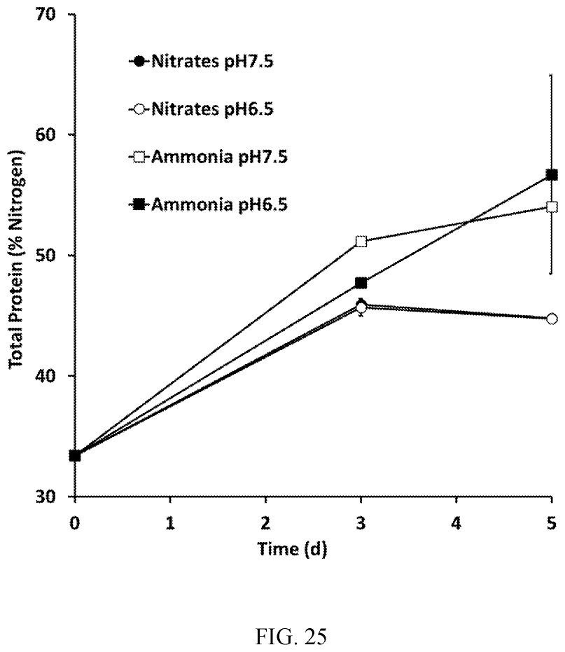

A shown in FIG. 22, the Ammonia pH 6.5 treatment produced the greatest amount of growth. As shown in FIGS. 23-24, the cultures did not deplete all of the available nitrogen, and the Ammonia treatments held the ammonia constant around 0.4 g/L. As shown in FIG. 25, the Ammonia treatments resulted in more protein than the Nitrate treatments. Therefore, the results show that utilizing an ammonium-pH auxostat system, microalgae growth rate and protein accumulation were able to be increased over the Nitrate treatment. Also, as an example, resulting protein content may be further increased by culturing the microalgae at a higher pH, such as one that is closer to the pKa of ammonia.

### Example 5

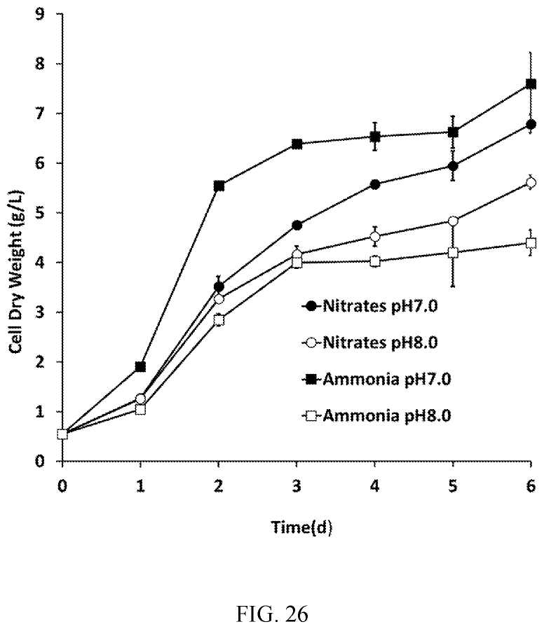

Another demonstration was undertaken to show that ammonia uptake may be induced in mixotrophic microalgae cells using an ammonium-pH auxostat system to increase growth and protein accumulation. Cultures of Chlorella (HS26) were inoculated at 0.3 g/L in glass column bioreactors at a volume of 700 mL of BG-11 culture media and maintained at a temperature of 27° C. The mixotrophic cultures received aeration at a rate of 1 Liter per minute, an initial batch of 30 g glucose/L, and were supplied 270 micromoles of light using LED lights (LumiGrow, Inc., Emeryville, Calif.). In a first treatment, the culture was supplied with an initial batch of 3 g/L NaNO3 (“Nitrate” treatment), and the culture pH was controlled at a set point of 7.0 by a pH auxostat feed containing 0.50% HCl. In a second treatment, the culture was supplied with an initial batch of 3 g/L NaNO3 (“Nitrates” treatment), and the culture pH was controlled at a set point of 8.0 by a pH auxostat feed containing 0.50% HCl. In a third treatment, the culture was supplied with an initial batch of 1.0 g/L NH4Cl, and the culture pH was controlled at a set point of 7.0 by a pH auxostat feed containing 0.25% NH4OH (“Ammonia” treatment). In a fourth treatment, the culture was supplied with an initial batch of 1.0 g/L NH4Cl, and the culture pH was controlled at a set point of 8.0 by a pH auxostat feed containing 0.25% NH4OH (“Ammonia” treatment). Samples were taken daily to measure the cell dry weight, nitrogen concentration, and total protein. Results are shown in FIGS. 26-29.

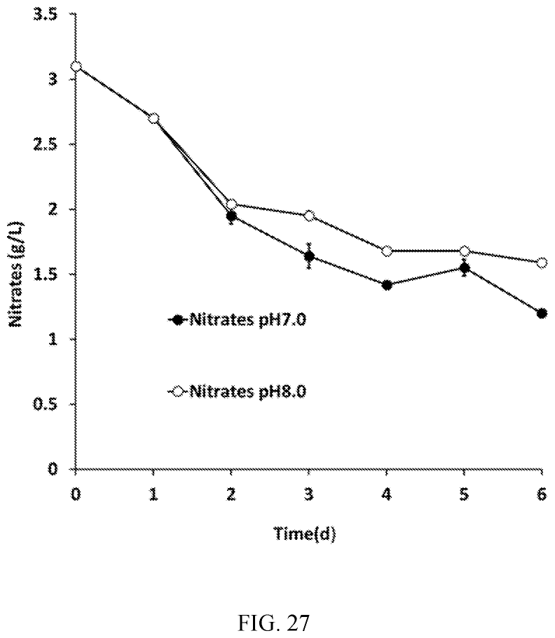

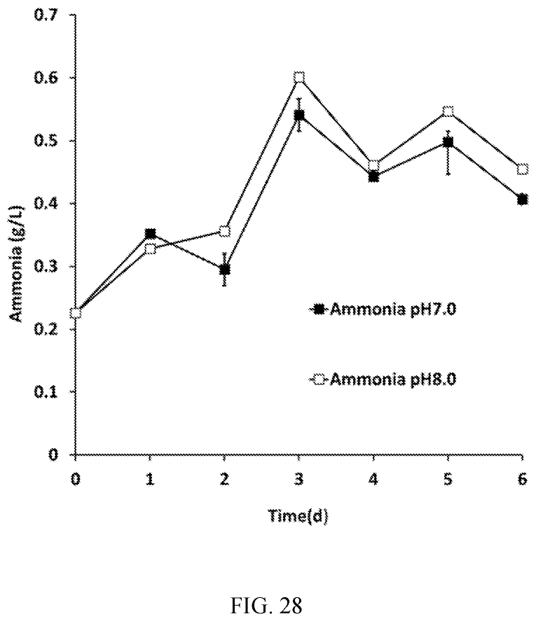

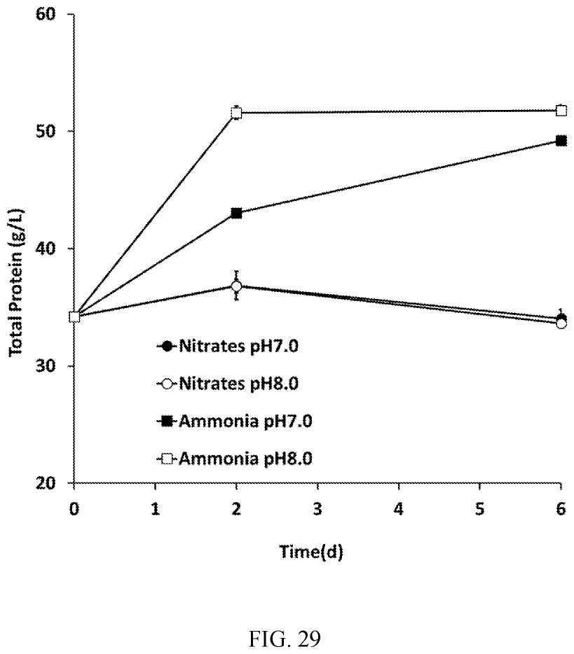

As shown in FIG. 26, the Ammonia pH 7.0 treatment had the highest resulting growth and the Ammonia pH 8.0 showed evidence of impaired growth (e.g., due to ammonia toxicity). As shown in FIGS. 27-28, the cultures did not deplete all of the available nitrogen. As shown in FIG. 29, the Ammonia treatments resulted in more total protein than the Nitrate treatments, with the culture grown at a higher pH (e.g., closest to the pKa level of ammonia, about 9.26) having the highest resulting protein. Therefore, the results of utilizing an ammonium-pH auxostat system microalgae illustrate that the growth rate and protein accumulation are able to be increased, when compared to Nitrate treatment. Also, as illustrated, the protein content can be further increased by culturing at a higher pH, such as one that is closer to the pKa of ammonia.

### Example 6

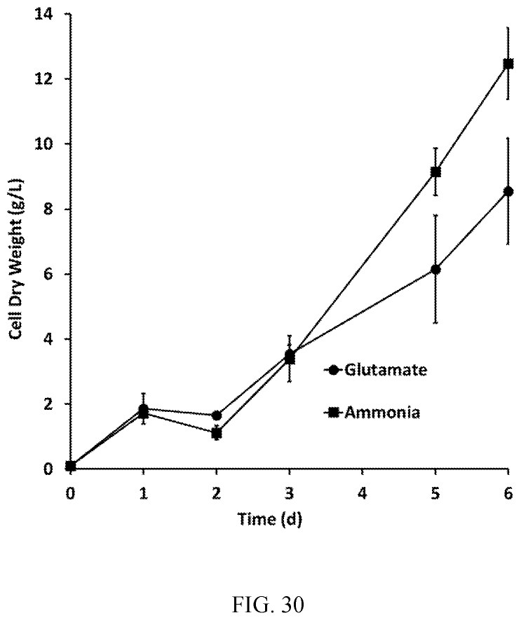

Another demonstration was performed to illustrate that ammonia uptake may be induced in heterotrophic microalgae cells using an ammonium-pH auxostat system to increase growth and protein accumulation. In this demonstration, cultures of Schizochytrium limacinum were inoculated at 0.1 g/L in glass column bioreactors at a volume of 700 mL of culture media and maintained at a temperature of 27° C. The heterotrophic cultures received aeration at a rate of 1 Liter per minute, and an initial batch of 80 g glycerol/L. In a first treatment, the culture was supplied with an initial batch of 20 g/L monosodium glutamate (“Glutamate” treatment), and the culture pH was controlled at a set point of 6.5 by a pH auxostat feed containing 1% HCl. In a second treatment, the culture was supplied with an initial batch of 1.0 g/L NH4Cl, and the culture pH was controlled at a set point of 6.5 by a pH auxostat feed containing 10% NH4OH (“Ammonia” treatment). Samples were taken daily to measure the cell dry weight, nitrogen concentration, and total protein. Results are show in FIGS. 30-33.

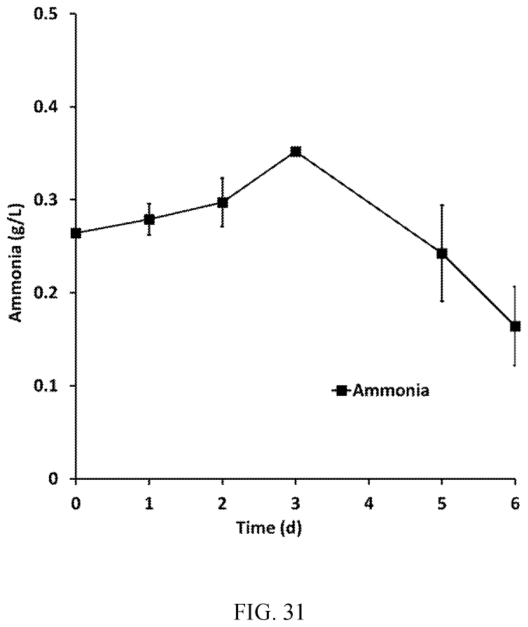

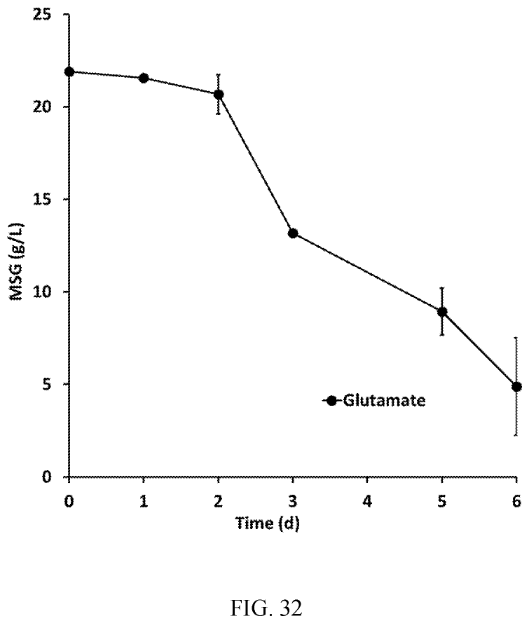

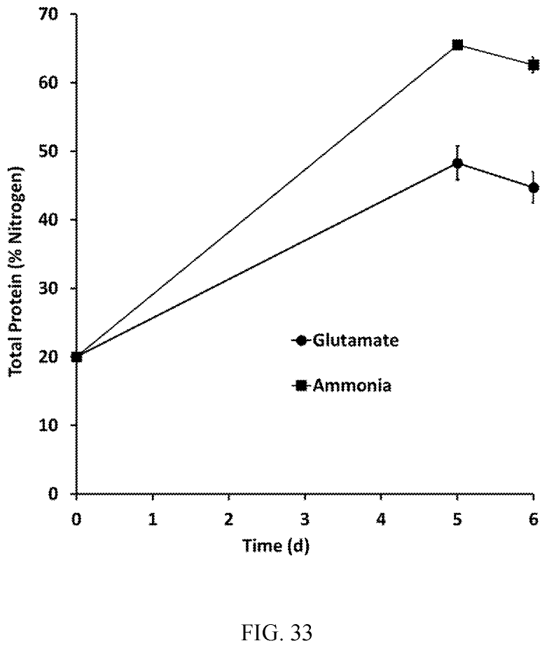

As shown in the FIG. 30, the Ammonia treatment had a higher resulting growth than the Glutamate treatment. As shown in FIGS. 31-32, the cultures did not deplete all of the available nitrogen. As shown in FIG. 33, the Ammonia treatment resulted in a 20% increase in protein yield over the Glutamate treatment. Therefore, utilizing an ammonium-pH auxostat system can result in an increased microalgae growth rate and protein accumulation.

### Example 7

As another example, it may be demonstrated that ammonia uptake may be induced in phototrophic microalgae cells using an ammonia as the nitrogen source, and carbon dioxide for pH control, to increase growth and protein accumulation. In this example, cultures of Chlamydomonas reinhardtii can be inoculated at 0.3 g/L in glass column bioreactors at a volume of 700 mL of BG-11 culture media and maintained at a temperature of 27° C. The phototrophic cultures can receive aeration at a rate of 1 Liter per minute and a supply of 270 micromoles of light using LED lights (LumiGrow, Inc., Emeryville, Calif.). The pH can be controlled with carbon dioxide and ammonium can be supplied as needed, as the nitrogen source. Treatments can include a culture pH of 7.0 and 8.0. Further, samples can be collected daily to measure the cell dry weight, nitrogen concentration, and total protein, as similarly described above.

### Aspects of the Methods and Systems Described Herein

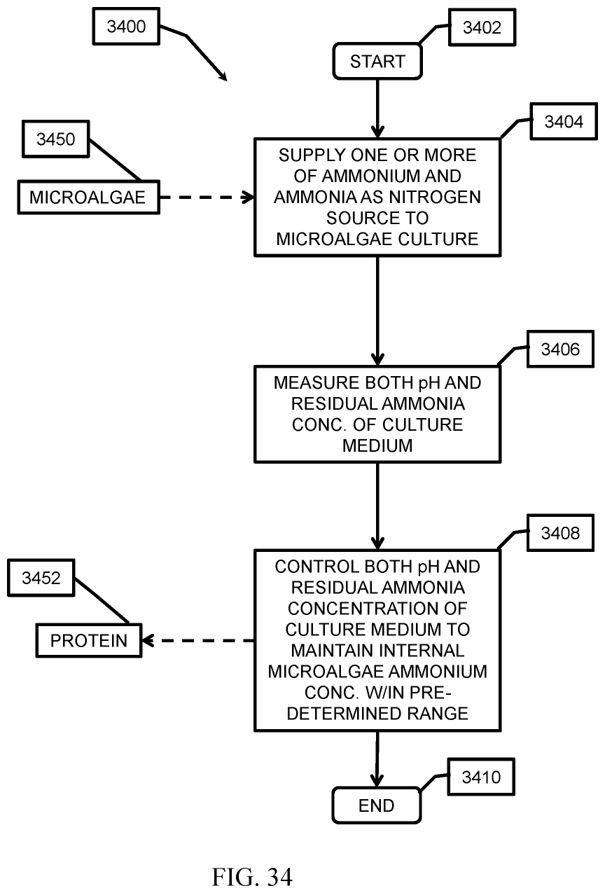

In one aspect, in a non-limiting embodiment, as illustrated in the flow diagram of FIG. 34, an exemplary method 3400 may be devised for managing ammonia toxicity for the benefit of a microalgae culture. In this example embodiment, the exemplary method 3400 can start at 3402. At 3404 a culture comprising a target microalgae 3450 (e.g., targeted for desired characteristics culture and/or production) can be supplied with at least one of ammonium and ammonia as a nitrogen source. At 3406, a pH of the culture medium can be measured, and a residual ammonia concentration in the culture medium can be measured. At 3408, the pH of the culture medium and the residual ammonia concentration in the culture medium can be controlled to maintain an internal microalgae cell ammonium concentration within a pre-determined range, based at least upon the measurements of the pH and residual ammonia concentration; resulting in an increase the protein content 3452 in the microalgae. At 3410, the exemplary method 3400 ends.

In some embodiments, the step of controlling the pH of the culture medium may further comprise the addition of NH4OH (ammonium hydroxide). In some embodiments, the NH4OH may be added as a titrant by a pH auxostat system. In some embodiments, the concentration of the NH4OH titrant may be in the range of 0.1-20%. In some embodiments, the concentration of the NH4OH titrant may be in the range of 0.1-1%. In some embodiments, the concentration of the NH4OH titrant may be in the range of 0.1-10%.

In some embodiments, the step of controlling the pH of the culture medium may further comprise the addition of a base comprising at least one selected from the group consisting of sodium hydroxide, magnesium hydroxide, and calcium hydroxide. In some embodiments, the method may further comprise supplying the microalgae culture with at least one organic carbon source selected from the group consisting of acetate, acetic acid, ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolyzate, proline, propionic acid, ribose, sacchrose, partial or complete hydrolysates of starch, sucrose, tartaric, TCA-cycle organic acids, thin stillage, urea, agricultural by-products, industrial process by-products, municipal waste streams, yeast extract, and xylose.

In some embodiments, the microalgae may be Chlorella. In some embodiments, the internal microalgae cell ammonium concentration may be maintained in the range of 2-10 mg/L. In some embodiments, the microalgae may be Aurantiochytrium. In some embodiments, the increase in protein may be at least 5% more compared to a culture receiving a nitrogen source that is not ammonium or ammonia. In some embodiments, the increase in protein may be up to 20% more compared to a culture receiving a nitrogen source that is not ammonium or ammonia.

In some embodiments, the pH of the culture medium may be controlled to maintain a pH in the range of 6.5-8.0. In some embodiments, the pH of the culture medium may be in the range of 6.5-7.0 and residual ammonia concentration in the culture medium may be less than or equal to 2.0 g/L. In some embodiments, the residual ammonia concentration in the culture medium may be in the range of 0.1-2.0 g/L. In some embodiments, the pH of the culture medium may be in the range of 7.0-7.5 and residual ammonia concentration in the culture medium may be less than or equal to 2.0 g/L. In some embodiments, the residual ammonia concentration in the culture medium may be in the range of 0.1-2.0 g/L.

In some embodiments, the pH of the culture medium may be in the range of 7.5-8.0 and residual ammonia concentration in the culture medium may be less than or equal to 0.5 g/L. In some embodiments, the residual ammonia concentration in the culture medium may be in the range of 0.01-0.50 g/L. In some embodiments, the pH of the culture medium may be in the range of 8.0-8.5 and residual ammonia concentration in the culture medium may be less than or equal to 0.5 g/L. In some embodiments, the residual ammonia concentration in the culture medium may be in the range of 0.01-0.50 g/L. In some embodiments, the method may further comprise supplying the microalgae culture with a supply of light comprising photosynthetically active radiation (PAR).

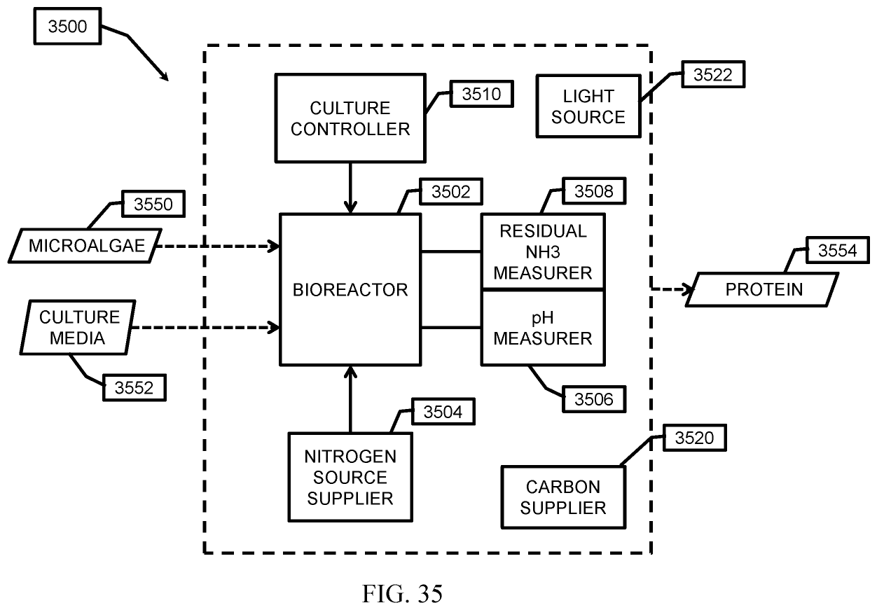

In one aspect, in one non-limiting embodiment, as illustrated in the schematic diagram of FIG. 35, an exemplary system 3500 may be devised for managing ammonia toxicity for the benefit of a microalgae culture. In this example embodiment, the exemplary system 3500 can comprise a bioreactor 3502 that is configured to culture a target microalgae 3550 in an appropriate culture media 3552. Further, the exemplary system can comprise a nitrogen source supplying component 3504 that is configured to supply the microalgae 3550 with at least one of ammonium and ammonia, as a nitrogen source. Additionally, a pH measurement component 3506 can be configured to measure the pH of the culture media 3552 during the culturing of the microalgae 3550. A residual ammonia concentration measurement component 3508 may be configured to measure the residual ammonia concentration of the culture media 3552 during the culturing of the microalgae 3550. In this embodiment, the exemplary system 3500 can comprise a culture control component 3510. The culture control component 3510 can be configured to control both the pH of the culture medium 3552 and the residual ammonia concentration in the culture medium 3552, based at least upon the measurements from the pH measurement component 3506 and the residual ammonia concentration measurement component 3508. Controlling the culture medium makeup can help maintain an internal microalgae cell ammonium concentration within a pre-determined range, which may result in an increase in protein content 3554 in the microalgae 3550.

In one implementation, the exemplary system 3500 can comprise an organic carbon source supply component 3520. In this implementation, the organic carbon source supply component 3520 can be configured to supply the microalgae culture with at least one organic carbon source selected from the group consisting of acetate, acetic acid, ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolyzate, proline, propionic acid, ribose, sacchrose, partial or complete hydrolysates of starch, sucrose, tartaric, TCA-cycle organic acids, thin stillage, urea, agricultural by-products, industrial process by-products, municipal waste streams, yeast extract, and xylose.

In another implementation, the exemplary system 3500 can comprise a light source 3522. In this implementation, the light source 3522 can be configured to supply the microalgae culture with a supply of light comprising photosynthetically active radiation (PAR).

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The citation and incorporation of patent documents herein is done for convenience only and does not reflect any view of the validity, patentability, and/or enforceability of such patent documents.

This inventive concepts described herein include all modifications and equivalents of the subject matter recited in the claims and/or aspects appended hereto as permitted by applicable law.

### REFERENCES

U.S. Pat. No. 8,759,073
US20150296752
WO2013121365A1
U.S. Pat. No. 3,615,654
US20150315538
D. Voltaolina, et al. Growth of Scenedesmus in artificial wastewater. Bioresource Technology 68 (1998) 265-268.
N. Liu, et al. Mechanisms of ammonium assimilation by Chlorella vulgaris F1068: Isotope fraction and proteomic approaches. Bioresource Technology 190 (2015) 307-314.
Ruiz-Martinez, et al. Effect of temperature on ammonium removal in Scenedesmus sp. Bioresource Technology 191 (2015) 346-349.
L. Wu, et al. The effects of nitrogen sources and temperature on cell growth and lipid accumulation of microalgae. International Biodeterioration & Biodegradation 85 (2013) 506-510.
N. Tam, et al. Effect of ammonia concentrations on growth of Chlorella vulgaris and nitrogen removal from media. Bioresource Technology 57 (1996) 45-50
M. Muro-Pastor, et al. Regulation of ammonium assimilation in cyanobacteria. Plant Physiology and Biochemistry 41 (2003) 595-603.
Jasti et al. Eur. J. Biochem. 36(6), 1990, pp. 827-836.

Although a particular feature of the disclosed techniques and systems may have been disclosed with respect to only one of several implementations, such feature may be combined with one or more other features of the other implementations as may be desired and advantageous for any given or particular application. Also, to the extent that the terms “including”, “includes”, “having”, “has”, “with”, or variants thereof are used in the detailed description and/or in the claims, such terms are intended to be inclusive in a manner similar to the term “comprising.”

This written description uses examples to disclose the inventive concepts, including the best mode, and also to enable one of ordinary skill in the art to practice the inventive concepts, including making and using any devices or systems and performing any incorporated methods. The patentable scope of the inventive concepts is defined by the claims, and may include other examples that occur to those skilled in the art. Such other examples are intended to be within the scope of the claims if they have structural elements that are not different from the literal language of the claims, or if they include equivalent structural elements with insubstantial differences from the literal language of the claims.

In the specification and claims, reference will be made to a number of terms that have the following meanings. The singular forms “a”, “an” and “the” include plural referents unless the context clearly dictates otherwise. Approximating language, as used herein throughout the specification and claims, may be applied to modify a quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Moreover, unless specifically stated otherwise, a use of the terms “first,” “second,” etc., do not denote an order or importance, but rather the terms “first,” “second,” etc., are used to distinguish one element from another.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances an event or capacity can be expected, while in other circumstances the event or capacity cannot occur—this distinction is captured by the terms “may” and “may be.”

The best mode for carrying out the inventive concepts has been described for purposes of illustrating the best mode known to the applicant at the time and enable one of ordinary skill in the art to practice the inventive concepts, including making and using devices or systems and performing incorporated methods. The examples are illustrative only and not meant to limit the inventive concepts, as measured by the scope and merit of the claims. The inventive concepts have been described with reference to preferred and alternate embodiments. Obviously, modifications and alterations will occur to others upon the reading and understanding of the specification. It is intended to include all such modifications and alterations insofar as they come within the scope of the appended claims or the equivalents thereof. The patentable scope of the inventive concepts are defined by the claims, and may include other examples that occur to one of ordinary skill in the art. Such other examples are intended to be within the scope of the claims if they have structural elements that do not differentiate from the literal language of the claims, or if they include equivalent structural elements with insubstantial differences from the literal language of the claims.

1. A method of increasing protein content in microalgae, comprising:
providing a culture of microalgae, the microalgae having an ammonia toxicity threshold level;
supplying the culture of microalgae with at least one of ammonium and ammonia as a nitrogen source;
measuring both a pH of the culture medium and a residual ammonia concentration in the culture medium; and
controlling both the pH of the culture medium and the residual ammonia concentration in the culture medium to maintain an internal microalgae cell ammonium concentration below the ammonia toxicity threshold level in order to increase the protein content in the microalgae.

2. The method of claim 1, wherein the step of controlling the pH of the culture medium comprises adding NH4OH as a titrant by a pH auxostat system, wherein the concentration of the NH4OH titrant is in the range of 0.1-20%.

3. (canceled)

4. (canceled)

5. The method of claim 2, wherein the concentration of the NH4OH titrant is in the range of 0.1-1%.

6. The method of claim 2, wherein the concentration of the NH4OH titrant is in the range of 1-10%.

7. The method of claim 1, wherein the step of controlling the pH of the culture medium comprises the addition of a base comprising at least one selected from the group consisting of sodium hydroxide, magnesium hydroxide, and calcium hydroxide.

8. The method of claim 1, further comprising supplying the microalgae culture with at least one organic carbon source selected from the group consisting of acetate, acetic acid, ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolyzate, proline, propionic acid, ribose, sacchrose, partial or complete hydrolysates of starch, sucrose, tartaric, TCA-cycle organic acids, thin stillage, urea, agricultural by-products, industrial process by-products, municipal waste streams, yeast extract, and xylose.

9. The method of claim 1, wherein the microalgae is Chlorella.

10. The method of claim 9, wherein the internal microalgae cell ammonium concentration is maintained at up to 10 mg/L.

11. The method of claim 1, wherein the microalgae is Aurantiochytrium.

12. (canceled)

13. (canceled)

14. The method of claim 1, wherein the pH of the culture medium is controlled to maintain a pH in the range of 6.5-8.5.

15. The method of claim 14, wherein the residual ammonia concentration in the culture medium is less than or equal to 2.0 g/L.

16. (canceled)

17. (canceled)

18. (canceled)

19. (canceled)

20. (canceled)

21. (canceled)

22. (canceled)

23. The method of claim 1, further comprising supplying the microalgae culture with a supply of light comprising photosynthetically active radiation (PAR).

24. A system for managing ammonia toxicity for the benefit of a microalgae culture, comprising:
a bioreactor to culture a target microalgae in an appropriate culture media;
a nitrogen source supplying component to supply the microalgae with at least one of ammonium and ammonia;
a pH measurement component to measure the pH of the culture media during the culturing of the microalgae;
a residual ammonia concentration measurement component to measure the residual ammonia concentration of the culture media during the culturing of the microalgae; and
a culture control component to control both the pH of the culture medium and the residual ammonia concentration in the culture medium to maintain an internal microalgae cell ammonium concentration within a pre-determined range to increase protein content in the microalgae.

25. The system of claim 24, the culture control component controlling the pH of the culture medium by adding NH4OH.

26. The system of claim 24, the culture control component comprising a pH auxostat system that adds a titrant.

27. The system of claim 24, the culture control component controlling the pH of the culture medium by adding a base comprising at least one selected from the group consisting of sodium hydroxide, magnesium hydroxide, and calcium hydroxide.

28. The system of claim 24, further comprising an organic carbon source supply component (3520) that supplies the microalgae culture with at least one organic carbon source selected from the group consisting of acetate, acetic acid, ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolyzate, proline, propionic acid, ribose, sacchrose, partial or complete hydrolysates of starch, sucrose, tartaric, TCA-cycle organic acids, thin stillage, urea, agricultural by-products, industrial process by-products, municipal waste streams, yeast extract, and xylose.

29. The system of claim 24, further comprising a light source that supplies the microalgae culture with a supply of light comprising photosynthetically active radiation (PAR).

30. The system of claim 24, wherein the culture control component controlling the pH of the culture medium to maintain a pH in the range of 6.5-8.0.

31. The system of claim 24, wherein the culture control component maintains the residual ammonia concentration in the culture medium at less than or equal to 2.0 g/L.
